(19) United States
(12) Patent Application Publication
(10) Pub. No.: US 2018/0127729 A1
(43) Pub. Date: May 10, 2018

(54) DLDH, DERIVATIVES THEREOF AND FORMULATIONS COMPRISING SAME FOR USE IN MEDICINE

(71) Applicant: RAMOT AT TEL-AVIV UNIVERSITY LTD., Tel Aviv (IL)

(72) Inventors: GIDEON FLEMINGER, Rehovot (IL); OSNAT ASHUR-FABIAN, Zur Moshe (IL); AVRAHAM DAYAN, Petah Tikva (IL)

(21) Appl. No.: 15/551,685

(22) PCT Filed: Feb. 17, 2016
(86) PCT No.: PCT/IL2016/050192

§ 371 Date: Aug. 17, 2017

(30) Foreign Application Priority Data
Feb. 17, 2015 (IL) 237283

(51) Int. Cl. C12N 9/02; A61K 38/44; A61K 45/06; A61K 41/00; A61K 33/24

(52) U.S. Cl. CPC C12N 9/0051; C12Y 108/01004; A61K 38/44; C07K 2319/70; A61K 41/0057; A61K 33/24; A61K 45/06

(57) ABSTRACT

The invention provides methods for treating a proliferative disease or disorder such as cancer by administering to patients suffering from the disease or disorder DLDH or a derivative thereof.

### TECHNOLOGICAL FIELD

The invention generally concerns novel methodologies for the treatment of cancer.

### BACKGROUND

Conventional therapeutic strategy in cancer is based on drugs that increase reactive oxygen species (ROS) generation and induce apoptotic cell death. These ROS moieties have been shown to selectively affect cancer cells but protect normal cells from ischemic damage. The protein Dihydrolipoamide dehydrogenase (DLDH) is critical for energy and redox balance in the cell. It has been reported in the literature that ROS are generated as a result of its oxidative activity [1].

WO 2004/113561 teaches the role of DLDH in developing of heart failure.

WO 2004/040298 discloses methods for the diagnosis and therapy of cancer by increasing the synthesis of proteins which are members of a protein-superfamily and which is associated with cell cycle regulation, cell mobility, oxidative stress response and protein fording, protein translocation and protein degradation.

U.S. Pat. No. 4,620,972 discloses a method of inhibiting the development of human cancer cells by administering to patient lactate dehydrogenase obtained from a primate or anti-lactate dehydrogenase obtained from a mammal, for inhibiting the lactate dehydrogenase activity in the cancer cells.

### REFERENCES

[1] Ralph S J, Moreno-Sánchez R, Neuzil J and Rodríguez-Enríquez S., Pharm. Res. 28, 2695-2730 (2011)
[2] Babor M, Gerzon S, Rahev B, Sobolev V, Edelman M., Proteins 70, 208-217 (2008)
[3] WO 2004/040298
[4] U.S. Pat. No. 4,620,972
[5] WO 2004/113561

### SUMMARY OF THE INVENTION

The inventors of the present application have now demonstrated that dihydrolipoamide dehydrogenase, DLDH, and derivatives thereof are effective as anti-cancer agents.

Cutaneous melanoma (CM) is one of the most rapidly growing cancers worldwide, with a consistent increase in incidence among white populations over the past four decades. CM is the most deadly form of skin cancer and an important public health concern, given the substantial health burden associated with the disease. The American Cancer Society estimates that skin cancer is the most prevalent of all cancers with over 2 million cases of nonmelanoma skin cancer each year and 75,000 melanoma cases in 2012. The rate of CM in the white population has dramatically increased during the last four decades mainely due to overexposure to sun UV irradiation.

The study leading to the present invention explored the treatment potential of CM as well as other proliferative disorders and cancers by a novel class of photocytotoxic nano-biocomplex developed by the inventors as a “neo-radiation” targeted therapy for such disorders. This novel nano-biocomplex, composed of TiO₂ nanoparticles conjugated with a specific TiO₂-binding protein bearing RGD (Arg-Gly-Asp) moieties, was designed to specifically recognize and bind members of the integrin family which are over-expressed in an array of aggressive cancer types, internalize via receptor-mediated endocytosis and to cause cancer cell damage upon illumination.

The cytotoxic effect of photo-excited titanium dioxide (TiO₂) by far UV (254 nm) illumination, creating reactive oxygen species (ROS), has been examined in several cancer models in vitro. However, serious damage to the surrounding healthy tissue limited the applicability of this approach. The development of the more appropriate treatment modality disclosed herein achieves TiO₂ photoxidative effect at the visible or near UV (>300 nm) range, causing less damage to the surrounding healthy tissue is desired.

The inventors have discovered a unique protein that strongly binds TiO₂. This protein, dihydrolipoamide dehydrogenase (DLDH) was known critical for energy and redox balance in the cell, yet was never used clinically in the treatment of cancers such as CM.

CM cancer cells as well as other cancer cells overexpress the cell surface receptor αvβ₃ integrin, which interacts with proteins of the extra cellular matrix through an RGD recognition site. The inventors have bio-engineered a novel class of materials based on human DLDH which serve as a bridge between the integrin expressing cancer cells and TiO₂ nanostructure forms. As TiO₂ has been shown to generate ROS-originated cytotoxic effects by itself, with particular effect on cancer cells, the inventors used this molecule to further produce a synergistic toxic effect leading to enhanced cancer cell death. This combinatory effect serves as a “neo-radiation” targeted treatment in a variety of cancers and other proliferative diseases.

As known in the art, dihydrolipoamide dehydrogenase “DLDH” is a homodimeric mitochondrial flavin-dependent oxidoreductase enzyme known to catalyze the NAD⁺-dependent oxidation of dihydrolipoic acid (or amide) into lipoic acid (or amide). NAD⁺ dependent metabolic and signaling pathways are highly altered in cancer cells. Thus, the enzyme's activity is critical for energy and redox balance in the cell and is often associated with elevated levels of reactive oxygen species (ROS) production.

While bioinformatics analysis indicates that the protein possesses a sequence and a structural homology with the apoptosis-inducing factor (AIF), a central player in apoptotic death, the involvement of DLDH in cytotoxicity, has never been predicted.

The inventors of the invention disclosed herein have now developed a methodology involving administering the mitochondrial enzyme to a living cell. As exhibited, the enzyme or a composition comprising the enzyme was found effective in treating a subject suffering or having a predisposition to suffering from a proliferative disease such as cancer. As demonstrated herein, the anti-cancer activity of DLDH, or a complex thereof with a metal oxide or an association product with a peptide, may be switched on by irradiation in order to maximize damage to cancer cells while reducing or diminishing damage to healthy cells.

Moreover, the inventors have isolated from a marine actinobacterium Rhodococcus rubber GIN1, a protein, TiBP, capable of strong adherence to TiO₂ and other metal and metal oxides, such as ZnO and magnetite. TiBP peptide mapping and sequencing revealed that TiBP is an exocellular form of DLDH. The strong, stable binding of DLDH and TiBP to TiO₂ was found to provide an excellent tool for serving as a bridge between cells and TiO₂ and other metal oxides. The novel approach of this invention for cancer therapy is based, among others, on combining the independent photoreactive ROS production capability of TiO₂ with that of DLDH and, with or without the targeting effect of the peptide RGD- when associated to DLDH, to produce a potent and selective anti-cancer therapy.

Thus, in one aspect of the invention, there is provided use of DLDH in medicine, e.g., in a method of treating a proliferative disease or disorder such as cancer.

As used herein, DLDH is used in its native form, a fragment, analog, homolog or derivative thereof, having the same biological characteristics or biological activity as the native DLDH, in the treatment of cancer. The source of the DLDH may be any prokaryote or eukaryote organism and it may be recombinant technology, peptide synthesis or biochemical isolation methods.

In some embodiments, the DLDH is isolated from an organism, for medicinal use; or may be prepared ex vivo or partially ex vivo, for a medical purpose.

In accordance with the invention, the DLDH may be used as is or may be used when modified, i.e., chemically modified, with at least one peptide, metal or metal oxide or any combination thereof.

In one aspect, the invention provides use of DLDH in the preparation of a composition for use in medicine, wherein said DLDH is an engineered DLDH or isolated DLDH. In some embodiments the DLDH may by purified DLDH.

The engineered DLDH may be genetically engineered. In some embodiments the DLDH is engineered by recombinant technology, peptide synthesis or biochemical isolation methods.

In one aspect, the invention provides use of DLDH in the preparation of a composition for use in medicine, wherein DLDH is provided by recombinant technology, peptide synthesis or biochemical isolation methods.

Thus, the invention generally provides an active material selected from DLDH and DLDH-based active materials selected from (1) DLDH associated with at least one peptide, (2) DLDH-metal ion or oxide complex, and (3) DLDH associated with at least one peptide as a complex with a metal ion or oxide, each of the active materials or any combination thereof being suitable for use in medicine, e.g., in the treatment or prevention of cancer.

In another aspect of the invention, the DLDH is provided as an association product with at least one peptide, and/or as a complex with at least one metal oxide.

In some embodiments, the DLDH is provided as an association product with at least one peptide.

In other embodiments, the DLDH is provided as a complex with at least one metal oxide.

In some embodiments, the DLDH is provided as an association product with at least one peptide, said DLDH further forming a complex with at least one metal oxide.

In a further aspect, the DLDH is provided as a peptide-modified material, the peptide comprising (or consisting) an integrin binding domain, e.g., arginine-glycine-aspartic acid (RGD).

The DLDH or DLDH-based active materials, as defined herein, are each formulated for use in medicine. Formulations or pharmaceutical compositions comprising each of the active materials may be further formulated for a particular use, e.g., treatment or prevention of a proliferative disease or disorder, such as cancer.

Thus, the invention further provides use of DLDH or a DLDH-based active material, as defined, in the preparation of a pharmaceutical composition for use in medicine.

In some embodiments, the pharmaceutical compositions are adapted for the treatment or prevention of a proliferative disease or disorder.

In some embodiments, the pharmaceutical compositions are adapted for the treatment or prevention of cancer.

The invention further provides a pharmaceutical composition or a medicament or a formulation for use in medicine, comprising DLDH or a DLDH-based active material, as defined herein.

A “pharmaceutical composition” refers to a preparation of DLDH or a DLDH-based active material, or physiologically acceptable salts or prodrugs thereof, optionally with a physiologically suitable carrier or excipient. The pharmaceutical compositions of the present invention may also include one or more additional active ingredients, such as, but not limited to, antibiotics, conventional anti-cancer or anti-inflammatory agents or any other active ingredient that may be suitable for combination therapy. Non-limiting examples of anti-cancer agents which may be used in combination with DLDH or a DLDH-based active material, and which may be included in a composition of the invention are antisense sequences, cis-platin and cis-platin derivatives or homologues; Tretinoin (Vesanoid®); interferon (IFN)-alpha; antineoplastics; Pegaspargase (Oncaspar®)); L-asparaginase; Edatrexate; 10-ethyl-10-deaza- aminopterin; 5-fluorouracil; Levamisole; Interleukin-2 (Proleukin®); Axcan; Methyl-chloroethyl-cyclohexyl-nitrosourea; Fluorodeoxyuridine; Vincristine; Porfimer Sodium (Photofrin®); Irinotecan (Camptosar®); Topotecan (Hycamtin®); Loperamide (Imodium®); Docetaxel (Taxotere®); Rituximab; Etoposide; Faulding; Vinorelbine Tartrate (Navelbine®); Paitaxel (Taxol®); Docetaxel (Taxotere®); Irinotecan; Gemcitabine; Gemcitabine (Gemzar®); Amifostine (Ethyol®); 2-ethylhexyl-p-methoxy-cinnamate; Prednisone (Deltasone®); Octyl-N-dimethyl-paminobenzoate; Benzophenone-3; Flutamide (Eulexin®); Finasteride (Proscar®); Terazosin (Hytrin®); Doxazosin (Cardura®); Goserelin Acetate (Zoladex®); Liarozole; Nilutamide (Nilandron®); Mitoxantrone (Novantrone®); Gemcitabine (Gemzar®); Porfimer Sodium; Dacarbazine; Etoposide; Faulding; Procarbazine HCl; Rituximab; Trastuzumab (Herceptin®); and Temozolomide (Temodal®).

The pharmaceutical compositions of the invention, for use in accordance with the present invention, may be formulated in a conventional manner using one or more pharmaceutically acceptable carriers comprising excipients and auxiliaries, which facilitate processing of the active materials into preparations which can be used pharmaceutically. Proper formulation is dependent upon the route of administration chosen.

The DLDH or a DLDH-based active material may be alternatively formulated into a delivery system such as encapsulation in liposomes, nanoparticles, microparticles, microcapsules or capsules, emulsions, dispersions and others, which may be used to administer the active material or compositions of the invention.

The compositions of the invention may be adapted for administration by one or more of the following administration routes: intradermal, intramuscular, intraperitoneal, intravenous, subcutaneous, intranasal, epidural, oral, sublingual, intranasal, intracerebral, intravaginal, transdermal, rectally, by inhalation, or topically to the ears, nose, eyes, or skin. The preferred mode of administration may be determined by the medical practitioner based, inter alia, on the site of the medical condition, the severity of the condition, the subject to be treated and other medical parameters.

The DLDH-based active materials according to the invention may also be utilized as photoreactive materials for a variety of other medicinal or non-medicinal applications and may thus be formulated based on their targeted use.

The active materials, as defined herein, may be administered alone in one therapeutic dosage form or in combination with at least one other anti-cancer drug or with generally at least one other active material, in two separate therapeutic dosages such as in separate capsules, tablets or injections. Also, the active materials, as defined herein, may be administrated in two separate therapeutic dosages, the administration may be such that the periods between the administrations vary or are determined by the medical practitioner. It is however preferred that the second drug (or second dosage) is administered within the therapeutic response time of the first drug (or first dosage).

Where the active materials are administered in combination with another drug or anti-cancer drug, they may be administrated simultaneously, namely together or separately but as a single treatment, or in sequence, in two separate therapeutic treatments. The two separate treatments may be administered for example one immediately after the other, or in any other regimen that the practitioner prescribing the combined treatment may find suitable based on the condition of the subject and any other parameters.

The active materials for use in accordance with the invention, may be administered in combination with at least one drug, being in some embodiments at least one anti-cancer drug, with a chemotherapy modality (treatment) or with radiation therapy. As is evident from the results presented herein, irradiation of the subject during, simultaneously with or subsequent to administering an active material according to the invention may not be necessary to achieve, enhance or otherwise modulate an anti-cancer effect. In some embodiments, however, irradiation may be useful in switching on the anti-cancer treatment, may be useful for enhancing the anti-cancer effect may be useful for dissociating an active material in order to render synergistic or more effective the anti-cancer effect.

In some embodiments, the active materials utilized according to the invention are administered irrespective and independent of irradiation therapy.

In some embodiments, where irradiation is involved, the subject or any region of the subject's body may be irradiated with UV source with a radiation flux which is preferably below the radiation flux considered safe by international standards. In some embodiments, the radiation is by UVA, UVB or UVC light, or by visible light.

In another aspect of the invention, there is provided a method of treatment or prevention of a disease or disorder, the method comprising administering to a subject in need thereof an effective amount of DLDH or a composition comprising same or a DLDH-based active material, as defined herein, or a composition comprising same, for the treatment or prevention of a disease or disorder.

In some embodiments, the disease or disorder is a proliferative disease or disorder. In some embodiments, said proliferative disease or disorder is cancer.

In some embodiments, the active ingredient for the treatment of cancer is DLDH. In some embodiments, the treatment involves administering DLDH to a subject in need of anti-cancer treatment, without necessitating radiation therapy; namely the treatment with DLDH does not involve radiation therapy at any stage of the treatment with DLDH.

In some embodiments, treatment with DLDH is provided in combination with radiation or chemotherapy.

As noted below, the use of DLDH in the treatment of cancer provides the opportunity to reduce the amount of at least one anti-cancer drug, chemotherapy or radiation when administered in combination with DLDH. Thus, in another aspect, the invention provides a method for reducing a dosage size or an effective amount of at least one anti-cancer drug administered to a subject at the onset or in the course of anti-cancer therapy, the method comprising administering DLDH and the at least one anti-cancer drug to said subject, the DLDH being in an amount sufficient to reduce the concentration or level of the at least one anti-cancer drug needed, while maintaining the same therapeutic effect as compared to administering the at least one anti-cancer drug alone.

In some embodiments, the anti-cancer drug is a drug known to be effective in the treatment of cancer. The anti-cancer drug may be an anti-cancer modality selected from chemotherapy and irradiation therapy.

In some embodiments, the anti-cancer drug is selected from antisense sequences, cis-platin and cis-platin derivatives or homologues; Tretinoin (Vesanoid®); interferon (IFN)-alpha; antineoplastics; Pegaspargase (Oncaspar®); L-asparaginase; Edatrexate; 10-ethyl-10-deaza-aminopterin; 5-fluorouracil; Levamisole; Interleukin-2 (Proleukin®); Axcan; Methyl-chloroethyl-cyclohexyl-nitrosourea; Fluorodeoxyuridine; Vincristine; Porfimer Sodium (Photofrin®); Irinotecan (Camptosar®); Topotecan (Hycamtin®); Loperamide (Imodium®); Docetaxel (Taxotere®); Rituximab; Etoposide; Faulding; Vinorelbine Tartrate (Navelbine®); Paitaxel (Taxol®); Docetaxel (Taxotere®); Irinotecan; Gemcitabine; Gemcitabine (Gemzar®); Amifostine (Ethyol®); 2-ethylhexyl-p-methoxy-cinnamate; Prednisone (Deltasone®); Octyl-N-dimethyl-paminobenzoate; Benzophenone-3; Flutamide (Eulexin®); Finasteride (Proscar®); Terazosin (Hytrin®); Doxazosin (Cardura®); Goserelin Acetate (Zoladex®); Liarozole; Nilutamide (Nilandron®); Mitoxantrone (Novantrone®); Gemcitabine (Gemzar®); Porfimer Sodium; Dacarbazine; Etoposide; Faulding; Procarbazine HCl; Rituximab; Trastuzumab (Herceptin®); and Temozolomide (Temodal®).

In some embodiments, where DLDH, or any DLDH-based active material, is provided as a peptide-modified material, comprising (or consisting) the peptide sequence arginine-glycine-aspartic acid (RGD), the RGD may be covalently associated with the DLDH protein through any one atom or site on the DLDH backbone. In some embodiments, the RGD is associated with the DLDH through the N terminus of the DLDH. In other embodiments, the RGD is associated with the DLDH through the C terminus of the DLDH. In further embodiments, the RGD is associated with the DLDH through both the N and C termini, providing an association product of DLDH with two RGD units (DLDH-RGD₂).

In some embodiments, DLDH is associated with at least one RGD. In some embodiments, DLDH is associated with at least two RGD. In some embodiments, DLDH is associated with at least three RGD. In some embodiments, DLDH is associated with a plurality of RGD units.

In some embodiments, the DLDH is modified on both ends of the molecule with RGD (DLDH-RGD₂) to provide a modified protein capable of specifically targeting integrin expressing cancer cells.

The DLDH-peptide association product (e.g., DLDH-RGD₂) may be utilized in a method for the treatment or prevention of cancer, the method comprising administering to a subject in need of an anti-cancer therapy an effective amount of the association product.

In some embodiments, the method of treatment or prevention does not comprise exposing the subject to an irradiation source following or concomitant with the administration of the association product. In some embodiments, the association product is administered to a subject prior to, after or simultaneously with chemotherapy and/or radiation therapy.

The invention further provides a method for reducing a dosage size of an at least one anti-cancer drug, the method comprising administering the at least one anti-cancer drug with an association product of DLDH with at least one peptide (e.g., DLDH-RGD₂), the association product being in an amount sufficient to reduce the concentration or level of the at least one anti-cancer drug needed, while maintaining the same therapeutic effect as compared to administering the at least one anti-cancer drug alone.

In some embodiments, the DLDH is provided as a complex with at least one metal or metal oxide (DLDH-metal or DLDH-metal oxide).

In some embodiments, the metal is selected from Ti, Fe, Zr, Zn, Cu, Ce and Al. In some embodiments, the metal is selected from Ti, Fe, Zr and Ce. In some embodiments, the metal is Ti.

The metal oxide is as oxide of a metal selected from Ti, Fe, Zr, Zn, Cu, Ce and Al. In some embodiments, the metal oxide is an oxide of a metal selected from Ti, Fe, Zr and Ce. In some embodiments, the metal is Ti and the metal oxide is TiO₂.

In some embodiments, the composition comprising the DLDH-metal oxide is administered following or simultaneous with irradiation. In some embodiments, the activity of the DLDH is modulated by irradiation.

In some embodiments, the pharmaceutical composition is adapted for use simultaneously with or prior to irradiation, wherein the irradiation causes dissociation of a bond between DLDH and said metal oxide, thereby activating DLDH for anti-cancer activity.

In some embodiments, the anti-cancer activity (cytotoxic effect) of said complex is higher than the anti-cancer activity of TiO₂ when administered free of DLDH in combination with irradiation. In some embodiments, the anti-cancer activity of said complex is higher than the anti-cancer activity of each of TiO₂ and DLDH when administered separately in combination with irradiation.

Thus, the invention further provides a method for enhancing the anti-cancer activity of a complex of DLDH with a metal oxide, the method comprising administering to a subject, simultaneously with or subsequent to administering said complex, radiation therapy, to thereby enhance the anti-cancer effect of said complex.

The invention further provides a method for modulating the anti-cancer activity of DLDH, said method comprising administrating to a subject a complex of DLDH with at least one metal oxide, prior to administering to said subject radiation therapy, wherein the radiation therapy enhances the anti-cancer effect. In some embodiments, the radiation therapy causes dissociation of a bond between DLDH and the at least one metal oxide, thereby activating DLDH for anti-cancer therapy. In some embodiments, each of DLDH and metal oxide induce an anti-cancer effect following dissociation from each other.

The invention further provides a method for delivering a metal oxide, e.g., TiO₂ to cancer cells, the method comprising contacting cancer cells in vivo or ex vivo with a complex of DLDH and at least one metal oxide, irradiating said cancer cells when in contact with the complex, to thereby cause said complex to dissociate, permitting free metal oxide to associate with the cancer cells.

In some embodiments, the complex is administered in vivo to a subject suffering from cancer and the subject or a region of the subject's body is subsequently irradiated.

In additional embodiments, cancer cells are contacted with the complex ex vivo. In some embodiments, DLDH in a complex with a metal oxide is associated with at least one peptide comprising the sequence arginine-glycine-aspartic acid (RGD). This active material, DLDH-RGD₂-TiO₂, is capable of selectively affecting cancer cells while protecting normal cells from ischemic damage. The novel approach disclosed herein for cancer therapy is based on combining the independent photoreactive ROS production capability of TiO₂ with that of DLDH and, together with the targeting effect of the RGD tails, to produce a potent and selective anti-cancer effect. Thus, the invention further provides a selective method for cancer treatment, wherein cancer cells are targeted and eradicated, while healthy cells are left substantially unaffected.

The invention therefore also contemplates a method for selectively targeting cancer cells, the method comprising chemically associating (forming a bond) an anti-cancer drug with RGD, the RGD being capable of association to the cancer cells or cancerous tissue without substantially associating to non-cancerous tissues.

In some embodiments, the anti-cancer drug is DLDH or DLDH-metal oxide.

Generally speaking, the DLDH-based active materials may be regarded as photoreactive agents suitable for use when administering photodynamic therapy (PDT). As known in the art, during PDT light is used to destroy abnormal cells in tumors. The light may be external to the body or organ or tissue or may be situated inside a body cavity or organ or next to a diseased tissue. In some embodiments, the light source is transcutaneously introduced to a desired internal treatment site through a surgical incision and left in place for an extended period of time, so that the light emitted can administer PDT to destroy abnormal cells that have absorbed the photoreactive active material.

Thus, prior to administering PDT, a photoreactive active material according to the invention is administered to the subject, to be absorbed preferentially, due to the existence of a targeting moiety, into the diseased cells at a treatment site that are to be destroyed by the therapy. The invention therefore provides photodynamic cancer therapies, the therapeutic methods involve administering to a subject in need thereof an effective amount of a photo- reactive DLDH-based active material selected from DLDH-RGD, DLDH-TiO₂ and DLDH-RGD₂-TiO₂, and irradiating said subject or a region of the subject's body in order to render active said photoreactive material.

The active materials used in accordance with the intervention are administered for the purpose of preventing or treating cancer in a subject having predisposition to suffering from cancer or to a subject already suffering from cancer. The term “prevention or treatment”, or any lingual variation thereof, refers to administering a therapeutic amount of a composition of the invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, to prevent the disease from occurring, or a combination of two or more of the above. The term also refers to prevention of the disease from occurring. The treatment regimen and specific composition to be administered will depend on the type of proliferative disease to be treated and may be determined by various considerations known to the medical practitioner.

Where the disease or disorder to be treated is cancer, the term “anti-cancer activity” refers to at least one of the following: decrease in the rate of growth of the cancer (i.e., the cancer still grows but at a lower rate); cease of cancer growth, i.e., stasis of the cancer tumor occurs, and, in some cases, the cancer tumor diminishes or is reduced in size. The term also concerns reduction in the number of metastasis, reduction in the number of new metastasis formed, slowing of the progression of the cancer from one stage to the other and decrease in angiogenesis induced by the cancer. In some embodiments, the cancer tumor is totally diminished. As stated above, this term also concerns prevention for prophylactic situations or for those patients susceptible to contracting cancer; the administration of said active materials will reduce the likelihood of the individuals contracting the disease.

In some embodiments, the term refers to any of cytotoxicity, necrosis and apoptosis.

Typically, an active material is administered in an “effective amount”, namely in an amount which is required to treat or prevent the disease or disorder in a clinically relevant manner A sufficient amount of the active material used to practice the invention for therapeutic treatment of conditions caused by or contributing to said disease or disorder varies depending, inter alia, on the manner of administration, the age of the patient, body weight, and the general health of the patient. The practitioners will decide the appropriate amount and dosage of the regimen.

The term “cancer” refers to all types of cancer or neoplasm or malignant tumors found in mammals, including melanoma leukemia, carcinomas and sarcomas. Examples of cancers are Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, ovarian cancer, cutanous melanoma, thyroid cancer, neuroblastoma, esophageal cancer, genitourinary tract cancer, malignant hypercalcemia, cervical cancer, endometrial cancer, adrenal cortical cancer and prostate cancer.

In some embodiments, the cancer is selected amongst cancers which over express an integrin receptor.

In some embodiments, the cancer is selected from ovarian cancer, cervical cancer and cutanous melanoma.

In some embodiments, the cancers are associated with integrin over expression. In some embodiments, said cancers are selected from brain cancer (e.g., Glioblastoma Multiforme, astrocytoma), breast cancer, colon cancer, gastric cancer, prostate cancer, pancreatic cancer, lung cancer, esophagus cancer, liver cancer, renal cancer, cancer of the urinary tract, bone cancer, bone marrow cancers, hematological cancers and tumors of the vacular endothelial cells.

In some embodiments, the disease or disorder to be treated is a proliferative disease, other than cancer. The proliferative disease is selected from a variety of infections, thrombosis, Psoriasis, Asthma, Multiple sclerosis, ulcerative colitis, Age-related macular degeneration and autoimmune disorders, growth of tissue, embryonic development and angiogenesis.

In further embodiments, the disease or disorder to be treated is selected from osteoporosis, Paget's disease, ovariectomy-induced physiological change, rheumatic arthritis, osteoarthritis and angiogenesis-related eye disease, diabetic retinopathy, corneal neovascularizing diseases, ischaemia-induced neovascularizing retinopathy, high myopia and retinopathy of prematurity,

In another aspect, the invention contemplates a peptide comprising at least one CHED [2] motif for medical use, as defined herein. As known, CHED is a structural motif on the surface of metal binding proteins which contains at least three of the following amino acid residues: cysteine (C), histidine (H), glutamic (E) or aspartic acid (D) and which forms strong coordinative and electrostatic bonds between the protein and the metal.

In some embodiments, the peptide consists of CHED.

In some embodiments, the peptide comprises CHED.

In some embodiments, CHED is associated with a metal or a metal oxide.

In some embodiments, the peptide comprising CHED is DLDH, or any DLDH-based active material as disclosed herein.

In another aspect, the invention provides DLDH for use as DNase.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D—present ESEM micrograms of: FIG. 1A—Anatase on metal plates; FIG. 1B—Anatase on glass covered TitanShield nano-tubes (NT); FIG. 1C—Anatase nano-particles (NP); FIG. 1D- Rutile on metal plates; FIG. 1E—Rutile micro-particles (μP); and FIG. 1F—P25-NP.

FIGS. 2A-B—present ROS production by TiO₂ anatse NP: FIG. 2A—presents ROS production by different amounts of the oxide after 30 min of UVA illumination. FIG. 2B—presents ROS production at different time intervals of UVA illumination by a constant amount of TiO₂ anatse (0.4 mg/ml).

FIGS. 3A-B—present ROS production by DLDH-RGD₂. FIG. 3A presents ROS production by 50 nM DLDH-RGD₂ after 30 min at UVA illumination or in the dark, in presence or absence of the substrates dl-dihydrolipoamide (DHL, (0.5 mM) and NAD⁺ analog acetylpyridine adenine dinucleotide (AcPyAD, 0.9 mM). FIG. 3B presents the time-dependent ROS production by increasing concentrations of DLDH-RGD₂.

FIG. 4—presents a binding curve for DLDH-RGD₂ to TiO₂ anatase nano particles (NP). Dotted line represents a non-linear regression curve (based on saturated bindind model, Prism-GraphPad).

FIGS. 5A-B—show the binding of DLDH-RGD₂ to TiO₂. TiO₂ rutile discs were placed in polystyrene plates and coated with DLDH-RGD₂ by 1 h incubation in 0.1M sodium bicarbonate buffer, pH 7.5, at room temperature. After washing, bound DLDH was detected by reaction with specific anti DLDH-antibodies followed by reaction with secondary gold labeled (12 nm) anti-IgG antibodies (FIG. 5A). FIG. 5B shows a control not containing DLDH.

FIGS. 6A-C present the effect of L-carboxylic acids on DLDH binding to TiO₂. FIG. 6A shows the binding of glutamate (black) and aspartate (grey) to TiO₂ NP (solid) and to DLDH-RGD₂-coated TiO₂ NP (striped).A sample of each amino acid (1.0 mg-ml⁻¹ in 1.0 ml of sodium bicarbonate buffer, pH7.5, containing 1.0M NaCl) was incubated for 1 h at room temperature with 30 mg of intact or DLDH-RGD₂-coated TiO₂ nanoparticles. Binding of the amino acid to TiO₂ was determined by a fluorescamine assay of samples withdrawn from the supernatants at various time intervals. FIG. 6B shows DLDH-RGD₂ binding to Glutamate-coated particles. DLDH (300 μg in the same buffer) was incubated with glutamate-coated TiO₂ nanoparticles (30 mg). FIG. 6C shows the inhibiting effect of glutamate (black) or aspartate (grey) on DLDH-RGD₂ binding to TiO₂ NP.

FIG. 7—presents enzyme activities of soluble (DLDH-RGD₂, squares) and TiO₂-adsorbed (TiO₂-DLDH-RGD₂, circles). Initial velocities of substrate reduction by the enzyme in solution were monitored by OD₃₆₃.

FIGS. 8A-C—show the time-dependent release of DLDH-RGD₂ from the complex with TiO₂ upon UVA illumination in vitro experiments. FIG. 8A shows in vitro results. FIG. 8B shows a similar effect in B 16F10 cells. The cells (10⁵ cells per well) were incubated with the complex and illuminated with UVA for up to 60 min The protein was prestained with fluorescamine and was monitored in the cells using confocal microscopy with Leica SPS. A & B are at Dark conditions, C & D are after UVA illumination; A & C—No protein added; B & D—8 μM represent the complex prepared with 80 μg/ml protein+11.6 mg/ml TiO₂. FIG. 8C shows quantitation by mean fluorescence intensity of wells B/60 (black, control) and D/60 (grey) of. FIG. 8B.

FIG. 9 shows the binding of DLDH-RGD₂ to various metal oxides in 0.1M ammonium bicarbonate buffer, pH 8.0, in the presence (solid lines) of 3.0M NaCl or in its absence (dashed lines). PZC values are shown.

FIGS. 10A-B show the effect of fluorescamine-labelling on DLDH activity. FIG. 10A shows the enzyme activity of DLDH-RGD₂ at different substrate concentrations. FIG. 10B shows the enzyme activity of the fluorescently-labeled protein at different substrate concentrations.

FIGS. 11A-B show TiO₂±DLDH-RGD₂ effect on HeLa morphology before/after UVC, 10 min FIG. 11A and FIG. 11B show results obtained in the dark. FIG. 11A and FIG. 11B show results obtained under UV illumination. FIG. 11B and FIG. 11D show results obtained in the presence of RGD₂-DLDH. FIG. 11A and FIG. 11C in the absence of RGD₂-DLDH.

FIG. 12 TiO₂±DLDH-RGD₂ effects on HeLa survival before/after UVC, 10 min. FIG. 12A shows % survival of the cells after UVC illumination or in the dark, in the presence or absence of RGD₂-DLDH. FIG. 12B and FIG. 12C shows FACS analysis of the cells described in FIG. 12A under UVC illumination (FIG. 12C) or in the dark (FIG. 12B).

FIGS. 13A-C show the cell survival after UVA illumination. FIG. 13A shows a diagram of the setup for the ROS assay with anatase NT-covered net FIG. 13B shows the cell survival of cells on anatase NT -covered net±DLDH-RGD₂ and Hela survival±UVA, 1 h FIG. 13C shows FACS analysis.

FIGS. 14A-B: FIG. 14A shows cell survival (HEK293—normal, cervical—Hela) with/without UVA illumination (1 h, 365 nm) after 48 h incubation. FIG. 14B shows cell membrane αvβ₃ integrin expirations by FACS analysis.

FIG. 15 shows dose dependence of B 16F10 death in presence or absence of TiO₂ tested by Confocal Microscopy (Leica SP5). The cell nuclei are stained by Draq5 (Far red).

FIG. 16 shows the cytotoxic effect on B16F10 cells of the TiO₂/protein complex at different ratios after 24 h, with/without 1 h of UVA illumination, by Leica SP5.

FIGS. 17A-C show B 16F10 cells incubated with different concentrations of DLDH-RGD₂ after 48 h of incubation. FIG. 17A show microscopic images of CM FIG. 17B shows FACS analyses of early and late apoptosis markers and FIG. 17C shows FACS analysis of cell Cycle.

FIGS. 18A-E show FACS analysis of 3 different human melanoma cell lines (A375, WM368, WM3314) and mouse melanoma (B16F10) treated with different concentrations of DLDH-RGD₂. FIG. 18A shows % surviving cell. FIG. 18B shows Early apoptosis. FIG. 18C shows Necrosis. FIG. 18D shows Late apoptosis. FIG. 18E shows shows cell membrane αvβ3 integrin expirations by FACS analysis.

FIG. 19A shows the cytotoxic effect on B 1 6F10 cells of the DLDH-RDG₂ during time (min), obtained by confocal microscopy by Leica SP5. White arrows point to the cytoplasm, yellow to the nuclei. FIGS. 19B-E show the cytotoxic effect of DLDH-RDG₂ during time (min) on B16F10 cells by following stained internal markers—Nuclei, DLDH, PI and PSIVA.

FIG. 20A shows the cytotoxic effect of DLDH-RGD₂ on cancer and normal cells by LSM510. FIG. 20B shows cell membrane αvβ₃ integrin expirations by FACS analysis.

FIG. 21 shows the dependence of DLDH-RGD₂ cytotoxicity on RGD. B16F10 cells were incubated with 1 μM of either fluorescamine-labelled DLDH-RGD₂ (FIG. 21A) or DLDH (FIG. 21B). FIG. 21 C shows solvent control. The confocal images were taken with Leica SP5.

FIGS. 22A-B show the inhibitory effect of free RGD on fluorescamine-labeled DLDH-RGD₂ penetration to HEK293B₃ cells. FIG. 22A shows fluorescamine-labelled DLDH-RGD₂ and FIG. 21B shows fluorescamine-labelled DLDH-RGD₂+free RGD.

FIGS. 23A-E show the incubation of OVCAR 3 (100,000 per well) with DLDH-RGD₂ (FIG. 23A), DLDH (FIG. 23B), ovalbumin (FIG. 23C), glycine (FIG. 23D) and fluorescamine alone (4 ug/0.25 ml of DMEM without FCS, FIG. 23E). The time (in sec) since addition of the protein to the cells is indicated in each figure. FIG. 23F shows cell membrane αvβ3 integrin expirations by FACS analysis.

FIGS. 24A-B show the degradation of ds-DNA phage λ by DLDH-RGD₂. Composition of each lane is shown in the table below. FIG. 24A—shows fragmented DNA and FIG. 24B shows intact DNA (0.15 ug-ml⁻¹ each). Substrates are DHL (0.5 mM) and NAD analog AcPyAD (0.9 mM). The buffer used was 0.1M sodium bicarbonate, pH7.4.

TABLE 1

| Lane(s) | DNA | DLDH-RGD₂ | Substrates |
|---|---|---|---|
| 1 | + | — | − |
| 2, 7 | + | — | + |
| 3, 10 | + | 1 μM | + |
| 4 | − | 1 μM | − |
| 5 | − | 10 μM | − |
| 6, 11 | + | 10 μM | + |
| 8 | + | 10 μM | − |
| 9 | + | 1 μM | − |
| 12 | − | — | − |

### DETAILED DESCRIPTION OF EMBODIMENTS

Dihydrolipoamide dehydrogenase (DLDH) is a homodimeric mitochondrial flavin-dependent oxidoreductase enzyme. It comprises an essential constituent of the 2-oxo acid dehydrogenase cycles which convert 2-oxo acids to the corresponding acyl-CoA derivatives. It catalyzes the NAD⁺-dependent oxidation of dihydrolipoic acid (or amide) into lipoic acid (or amide). Interestingly, NAD⁺ dependent metabolic and signaling pathways are highly altered in cancer cells. The enzyme's activity is critical for energy and redox balance in the cell and is often associated with elevated levels of Reactive Oxygen Species (ROS) production. Additionally, bioinformatics analysis indicates that the protein possess sequence and structural homology with the apoptosis-inducing factor (AIF), a central player in apoptotic death. These characteristics make DLDH a relevant potential anti-cancer molecule for the selective cytotoxicity of cancer cells. However, involvement of DLDH in cytotoxicity has never been reported.

Integrins are a family of cell surface receptors which are over-expressed on all tumor vascular cells and an array of cancer types. Twenty-four integrin heterodimers are currently identified and formed by the combination of at least 18 α-subunits and 8 β-subunits. These integrin receptors play a key role in the cross-talk between the cell and its surrounding stroma, binding to ECM ligands, cell surface ligands and soluble ligands. A subset of these 24 integrins, including αvβ₃ integrin, interact with proteins of the extra cellular matrix through an RGD recognition site and offer a docking site for endothelial cells, inflammatory cells and cancer cells. αvβ₃ plays a pivotal role in cancer pathogenesis and is intensively studied. Besides its mechanical function, this integrin is a true signaling molecule which participates in activation of cell migration, survival and angiogenesis and communicates with an array of growth factor receptors in cancer, such as tyrosine kinas to trigger tumorigenesis. The high expression of this integrin, to such an extent that new imaging approaches based on its expression are now under development is of clinical significance as correlation with tumor progression in several cancer types has been documented. Therefore utilizing RGD-recognition integrins, such as αvβ₃, are attractive and rational targets for cancer treatment.

The inventors have bio-engineered a recombinant human DLDH with tails of the integrin binding domain, arginine-glycine-aspartic acid (RGD), on both ends of the molecule (DLDH-RGD₂) and generated a protein capable of specifically targeting integrin expressing cancer cells.

Titanium (Ti) is a powerful biocompatible material which is extensively being used in medical biomaterials applications e.g. in implantation. The oxide layer formed on the surface upon exposure to air, is important for protein adsorption which is considered to be an initial step in induction of differentiation of bone cells. Of the three naturally occurring crystallographic forms of titanium dioxide (TiO₂), anatase, rutile and brookite the former possesses highest photocatalytically activity which results from its higher hydrophilicity. Its high photoreactivity, physical stability and commercial availability as well as its low toxicity make TiO₂ the material of choice as a detoxifier which destroys cells, bacteria and organic toxic materials which is widely used for biomedical treatments including the destruction of cancer cells. The cytotoxic effect derived from ROS production of upon photo-excitation of TiO₂ by far UV, has been examined in several cancer models in vitro. However, serious damage to the surrounding healthy cells limits the applicability of this method. Thus, developing a technique that will achieve TiO₂ photo-oxidative effect at the visible or near UV (>351 nm) range as well as delivering the TiO₂ selectively to the cancer cells are desired. When TiO₂ is encountered with a human tissue it is rapidly covered with plasma and extra cellular matrix (ECM) proteins which strongly affect the biorecognition process. The adherence of TiO₂ to most proteins is dominated by weak, reversible, electrostatic or hydrophobic bonds which often results in failure in achieving strong attachment of cells and tissues.

Previously, a cell wall protein capable of strong adherence to coal fly ash (CFA) and TiO₂ particles, expressed solely during the late logarithmic phase of growth, has been isolated in our laboratory from the marine actinobacterium Rhodococcus rubber GIN1 and designated TiBP. The protein/oxide interaction occurred at high salt concentrations and its release from the oxide required high concentrations of SDS/urea, indicating non-electrostatic mechanism of binding, presumably via coordinative bonds. This is in contrast to most proteins that bind TiO₂ via relatively weak charge related interactions. Peptide mapping and sequencing revealed that TiBP is an exocellular form of DLDH. Docking analysis experiments performed by our group have led to identification of a putative TiO₂ binding site (CHED motif) on the protein molecule.

Conventional therapeutic strategy in cancer is based on drugs that increase ROS generation and induce apoptotic cell death. These ROS moieties have been shown to selectively affect cancer cells but protect normal cells from ischemic damage. The novel approach disclosed herein for cancer therapy is based on combining the independent photoreactive ROS production capability of TiO₂ with that of DLDH and, together with the targeting effect of the RGD tails, to produce a potent and selective anti-cancer effect. For the proof of concept the inventors have studied each of the components of the complex, individually or combined, in three cancer cell models: ovarian cancer, cervical cancer and cutanous melanoma, all over-expressing RGD-recognizing integrins, such as αvβ₃. DLDH itself is proposed to serve as a novel therapy and that illumination of the complex (DLDH-RGD₂-TiO₂) will produce high synergistic ROS activity and cell death and may serve as a “neo-radiation” targeted treatment in cancer.

It is further suggested that short metal-oxide motifs identified within the DLDH sequence, may be used to achieve a short peptide that can maintain the titanium oxide binding capabilities with high affinity and may also be effective in producing ROS and cancer cell death upon illumination.

Experimental

Materials

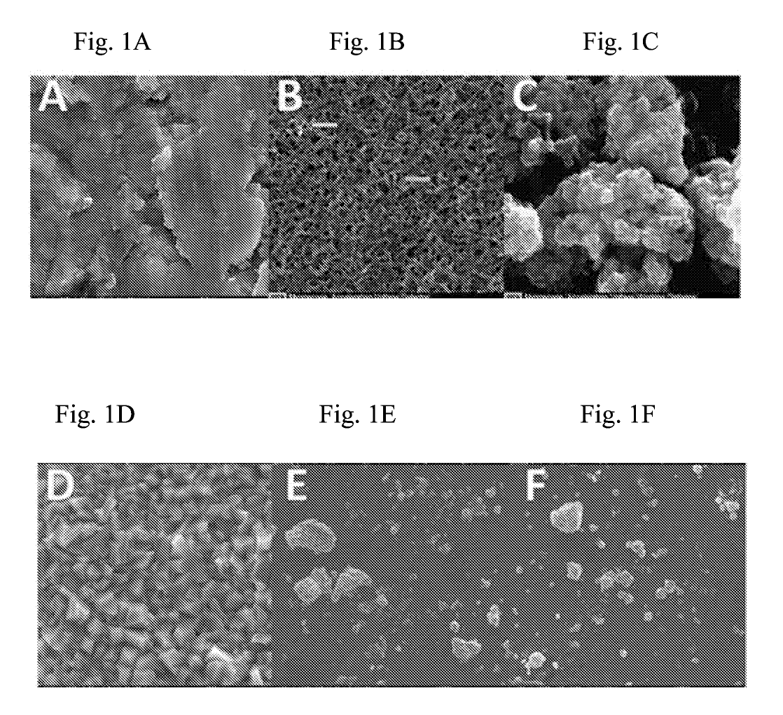

All reagents used in this study were of analytical grade and, unless otherwise specified, were purchased from Merck (Darmstadt, Germany) or Sigma-Aldrich (St. Louise, Mich.). Several TiO₂ forms were used as carriers in the present study: (1) anatase nano- and microparticles (Sigma, 23 and >100 nm, respectively) (2) TitanShield Colloid solution which form 8 nm particle net on glass disks and (3) oxidized—rutile, prepared by 5 h heating at 850° C. and anatase, prepared by 2 h heating at 450° C. (FIG. 1). The crystallographic forms and the metal composition of these preparations were confirmed by XRD, EDS and XRF (data not shown).

DLDH Cloning

Calcium chloride treated competent Escherichia coli BL21 (DE3) cells harboring the expression vector were aerated at 37° C. in Terrific Broth media, supplemented with 25 mg-ml⁻¹ kanamycin for 16 h. The cells were harvested, resuspended in 50 mM sodium phosphate buffer, pH7.5 and sonicated in the presence of 10 mg-ml^{-l} DNase E. and protease inhibitor cocktail (Sigma-Aldrich, St. Louise, Miss.). After sonication it was centrifuged (20,000 g for 30 min at 4° C.) and the supernatant fluid was collected and used for further purification. Recovery of the native form of the enzyme in the soluble fraction was indicated by its yellow color originating from the FAD prostatic group, as well as its enzymatic activity.

Lysozyme and diaphorase were purchased from Sigma-Aldrich. A pET28b-WT-DLD plasmid carrying the human dldh gene encoding DLDH (UniProt P09623), excluding the N-terminal 1-35 signal peptide region and containing an N terminal His₆ tag (kindly provided by Prof. Grazia Isaya from the Mayo clinic college, Rochester, Minn.) was transformed into competent E. coli BL21 cells.

Protein Purification

The expressed His-tagged protein was isolated by immobilized metal affinity chromatography (IMAC). The supernatant of the cell extract was loaded onto a Fast-Ni Column (5 ml, GE Healthcare, Upsalla), connected to Akta Chromatographic System (GE Healthcare, Upsalla). The column was washed with the washing buffer (50 mM potassium phosphate buffer, pH 6.0, containing 300 mM NaCl, 10% glycerol and 20 mM imidazole) at a flow rate of 2 ml-min⁻¹, until no protein was detected by OD₂₈₀. The His-tagged protein was then eluted with the elution buffer (0.5M imidazole in washing buffer, pH 6.0). The fractions were pooled and dialyzed against 0.1M sodium bicarbonate buffer, pH 7.5, at 4° C. for 16 h. The pooled protein fraction was further purified by gel filtration chromatography on a Superdex 200 column (300×10 mm, Akta Chromatographic System, GE Healthcare, Upsalla) collecting 1 ml fractions at a flow rate of 1 ml-min⁻¹ and analyzed by SDS-PAGE (12%) as commonly used. The gels were stained with Coomassie Blue R250. A Precision Plus Protein Standards Dual Color (Bio-Rad) markers mixture was used.

Determination of Protein and Peptide Concentrations

Protein concentrations were determined by absorbance at 280 nm using extinction coefficients of 0.479 calculated from the amino acid compositions of DLDH-RGD₂ (by the Expasy ProtParam application http://web.expasy.org/protparam/) or by the Bradford assay as commonly used

Oxide-Binding Assays

TiO₂ binding activity was determined by incubation of DLDH-RGD₂ or lysozyme with TiO₂ (Anatase) nanoparticles at a ratio of 10-15 μg protein per mg of beads in the indicated buffer. After agitation for 1 h at room temperature, the beads were sedimented by centrifugation in an Eppendorf (Hamburg, Germany) centrifuge for 15 min at 11000 g. The concentration of the non-adsorbed protein in the supernatant was determined as mentioned above.

Binding of aspartate and glutamate to native TiO₂ or to DLDH-RGD2-coated TiO₂ was determined by incubation for 1 h at room temperature of the particular amino acid (1.0 mg-mL⁻¹) with 30 mg of TiO₂ or TiO₂-DLDH-RGD2 (Anatase) nanoparticles in 1.0 mL of sodium bicarbonate buffer, pH 7.5, containing 1.0M NaCl. Binding of amino acids to the TiO₂ particles was determined by fluorescamine assay of samples withdrawn from the supernatants at various time intervals.

Enzyme Activity Assays

The substrate dl-dihydrolipoamide (DHL) was prepared by reduction of dl-lipoamide with sodium borohydride. Briefly, a suspension of 200 mg dl-lipoamide in 4 ml methanol and 1.0 ml of 2× distilled water, was cooled to 0° C. and stirred while dripping a cold solution (1 ml) of sodium borohydride (200 mg-ml⁻¹ in 2× distilled water) until the solution became clear and colorless. The solution was then acidified with dilute hydrochloric acid to pH˜2 and extracted with 5 ml chloroform. The chloroform extract was dried and evaporated in a desiccator. The residual material was crystallized from hexane/benzene (1:2.5). The product was recovered by centrifugation for 5 minutes at 4° C. (3800 g) using a Heraeus Megafuge 1.0 centrifuge (ThermoFisher Scientific Inc., Waltham, Mass.). The precipitate was air-dried, and stored at −20° C. Before used, the substrate was dissolved in acetone at a concentration of 120 mM.

DLDH-RGD₂

The reaction buffer contained 0.9 mM of the NAD⁺ analog acetylpyridine adenine dinucleotide (AcPyAD) in 2.5 mM sodium phosphate buffer, pH 7.6 containing 1.0 mM EDTA and the substrate DLH (0.1-0.5 mM). The reaction was initiated by addition of DLDH-RGD₂ (10 μl of 2 mg-ml⁻¹) into 1 ml of reaction buffer. Reduction rate of AcPyAD was continuously monitored by the absorbance at 363 nm. Activity was expressed as product produced (mM-min⁻¹), based on an extinction coefficient of a 9.1×10³M⁻¹ cm⁻¹ of AcPyAD.

TiO₂-DLDH-RGD₂

Bound DLDH-RGD₂ was prepared by binding samples of 20 μg of DLDH-RGD₂ to 10 mg of TiO₂ (Anatase) nanoparticles (100% bound) as described above. Prior to the experiment the particles were thoroughly washed with the assay buffer, excluding the substrate DHL, to remove any unbound enzyme. 10 mg of beads were mixed with 1.0 ml assay buffer, excluding the DHL substrate. Activity of the oxide-bound enzyme was determined under the above described conditions for the enzyme in solution. Since continuous, on-line monitoring was not possible due to the suspension turbidity, a discontinuous assay was applied as follows: The beads were agitated with 1.0 ml of the reaction buffer (excluding DHL) for 1 min and then sedimented by short centrifugation. The absorbance of the supernatant at 363 nm was monitored and used as reference. The beads were then re-suspended in the same buffer and the enzymatic reaction was initiated by addition of DHL (0.05-0.5 mM). After 1 min agitation the beads were sedimented and the absorbance at 363 nm of the supernatant was measured again. This process was repeated 5 times. Only the incubation times of the beads with the reaction mixture were taken into account in the activity assay. Incubation of the DLDH-RGD2 carrying beads in the reaction mixture without DHL served as a reference. Activity was defined as the ΔOD₃₆₃-min⁻¹ between the absorbance measurement and the former one.

ROS Generation Assay

The detection of ROS generated by A-NP activity is based on the reduction of Fe⁺³ to Fe⁺²-cytochrome C. This test was performed under UVA illumination for 30 min After incubation, the absorption of the liquid measured by spectrophotometer at a wavelength of 550 nm (E_{M 550 nm}=2.1×10⁴ M⁻¹ cm⁻¹).

ROS generation assay in vitro was carried out by cytochrome C reduction.

Photocatalysis Assay

Determination of the photocatalytic effect was made by photo-degradation of Methylene Blue and Degradation of Methyelene Blue by spectrum colorimetric assay.

The Cytotoxicity Effect

The different complex components, separately and combined, examined in vitro at various concentrations in three cancer cell models (ovarian cancer, cervical cancer and cutaneous melanoma) in cultures (24 wells) as well as in control cells (integrin positive; CV-1 cells and integrin negative; HEK293 cells) in the presence/absence of a selected optimal protocol of illumination and will be assessed for: Cell viability (WST-1, ELISA), Absolute cell number (FACS), Cell cycle (PI, FACS) and Cell death (Annexin-PI, FACS). Confocal microscopy (LSM 510, Leica SP5) used to analyze the interaction of the biocomplex with the cells and its internalization process.

Dyes—Draq5, Heuaecst, Fluorescamin, Annexin, PI, Psiva

Fluorescamine Labelling of Proteins

Protein, (typically 1-20 mg in 0.1M sodium carbonate buffer, pH 7.5-8.0, 1.0 ml) was mixed with 0.2 ml of 1M sodium borate buffer, pH9.5. Then 0.1 ml of fluorescamine (0.1 mg-ml⁻¹ of acetone) was added, and thoroughly Vortexed for about 15 sec.

Cell lines: Human ovarian adenocarcinoma cells were OVCAR-3 (ATCC HTB-161). Human cervical cancer (HeLA) and human melanoma cells (WM3314, A375, WM3682, WM3526) and mice melanoma (B16F10). Human normal embryonic kidney cells, HEK 293 (ATCC CRL1573) serve as healthy controls. The cells were cultured in RPMI1640 supplemented with 10% heat-inactivated FBS and antibiotics.

Flow cytometry: For absolute cell number, the cells were harvested in a fixed volume and counted. For Annexin-PI assay, cells were harvested and incubated with Annexin v-FITC and PI (BioVision) according to manufacture instructions and analyzed by flow cytometry. Annexin-/PI-, surviving cell fraction; Annexin+/PI-, early apoptosis; and Annexin+/PI+, late apoptotis. For cell cycle, the cells were permeablized by 70% ethanol and PI was added and the cells were analyzed by FACS.

Results

1. Characterization of the Individual Component of the TiO₂-DLDH-RGD₂ Compex

1.1 TiO₂ Preparations

Photoreactivity Upon UVA and UVC Illumination

The various TiO₂ preparations described in FIG. 1, were tested for MB degradation capability under both UVC and UVA illumination. As summerize in Table 2, highest activities were obtained with the anatase nanoparticles of Sigma.

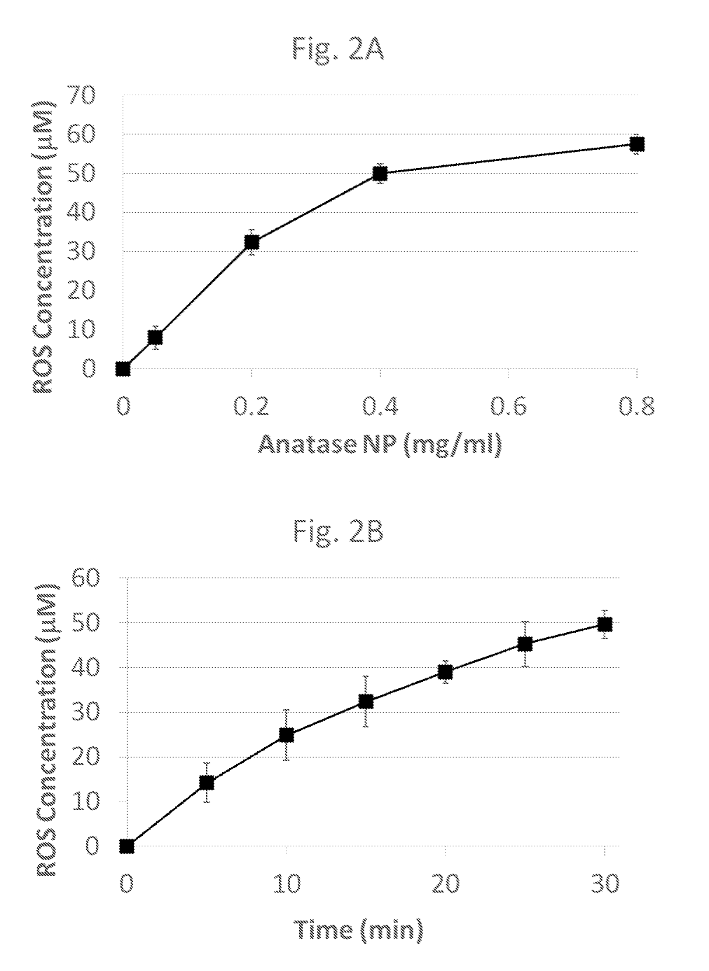

Production of Reactive Oxygen Species (ROS) which is the main cause for photodegradation was then analyzed by a specific in vitro assay based of Cyt C reduction for most efficient TiO₂ form in Table 2, Anatase NP preparation as shown in FIG. 2A and FIG. 2B.

1.2 DLDH-RGD₂

DLDH-RGD₂-recombinant DLDH with RGD tails on both termini prepared in E.coli and obtained as an active, 2×55 kDa holo(FAD) dimeric enzyme, as shown by spectrophotometry and FPLC analysis. Neither the TiO₂-binding properties nor its enzymatic activity DLDH were affected by the addition of the RGD tails (data not shown). It is pertinent to note that DLDH is enzymatically active only as a dimer and only when FAD is bound to the molecule.

ROS Production By DLDH-RGD₂

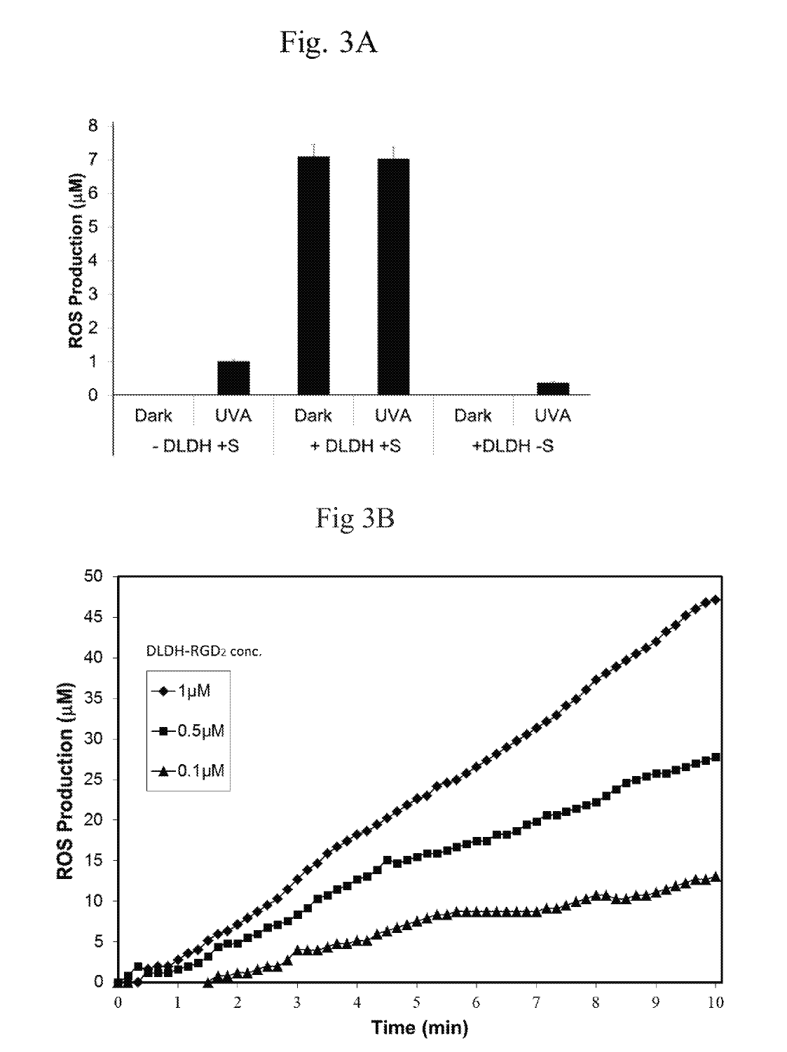

It was anticipated that the ROS generating ability of DLDH-RGD₂ should be associated with redox enzyme activity, an effect which was reported before to occur in the mitochondria of the living cells. The ROS generating activity was measured in the presence of the two co-substrates, DHL and the NAD analog (AcPyAD) or their absence, under UVA illumination or in the dark (FIG. 3A). The enzyme was found to be stable under UVA illumination and ROS production conditions (data not shown). The Figure indicates that DLDH-RGD₂ produces a similarly high ROS activity under dark and UVA conditions provided that the substrates are present. Next, ROS activity was measured by increasing concentrations of DLDH-RGD₂ at different incubation times (FIG. 3B). As shown in this Figure, ROS generation increases by DLDH-RGD₂ in a time dependent manner. Comparable ROS production was observed by the different DLDH-RGD₂ concentration (0.1-1 μM). This led us to further study the activity of DLDH-RGD₂ in vitro.

1.3 TiO₂-DLDH-RGD₂

Binding of DLDH-RGD₂ to TiO₂

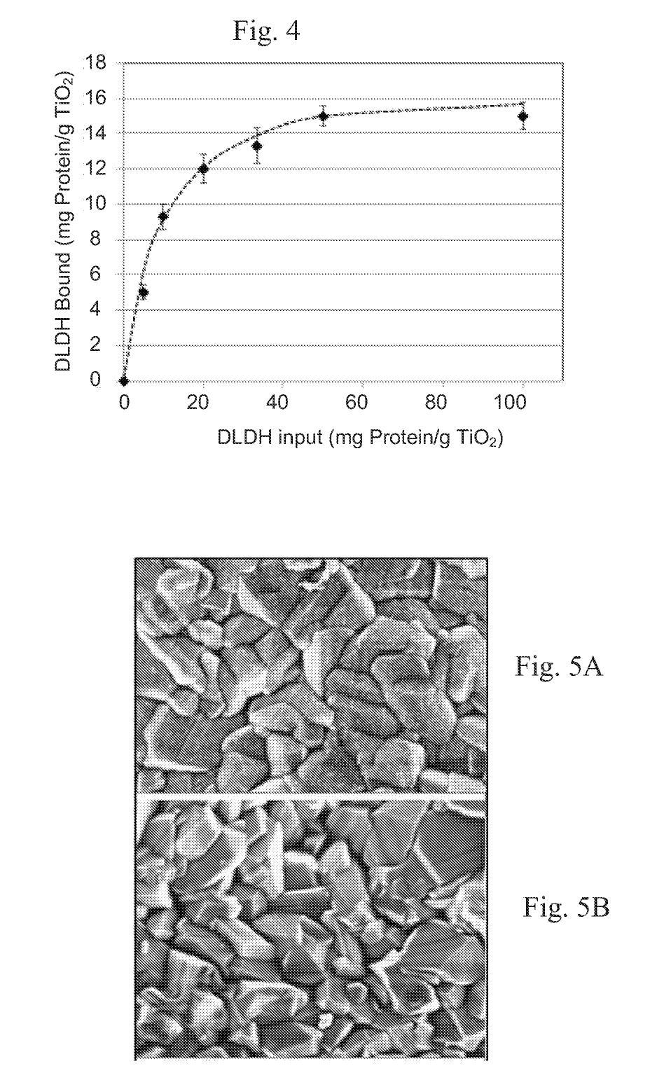

The binding isotherm of DLDH-RGD₂ to TiO₂ was analyzed next. A constant amount of the protein was incubated with increasing amounts of TiO₂ particles and the amounts of bound protein were determined. As shown in FIG. 4 a saturation curve was obtained with an apparent Kd of 9.43±1.38 mg DLDH-RGD₂ per g TiO₂ and a Bmax of 17.15±0.67 mg DLDH-RGD₂ bound per g of TiO₂.

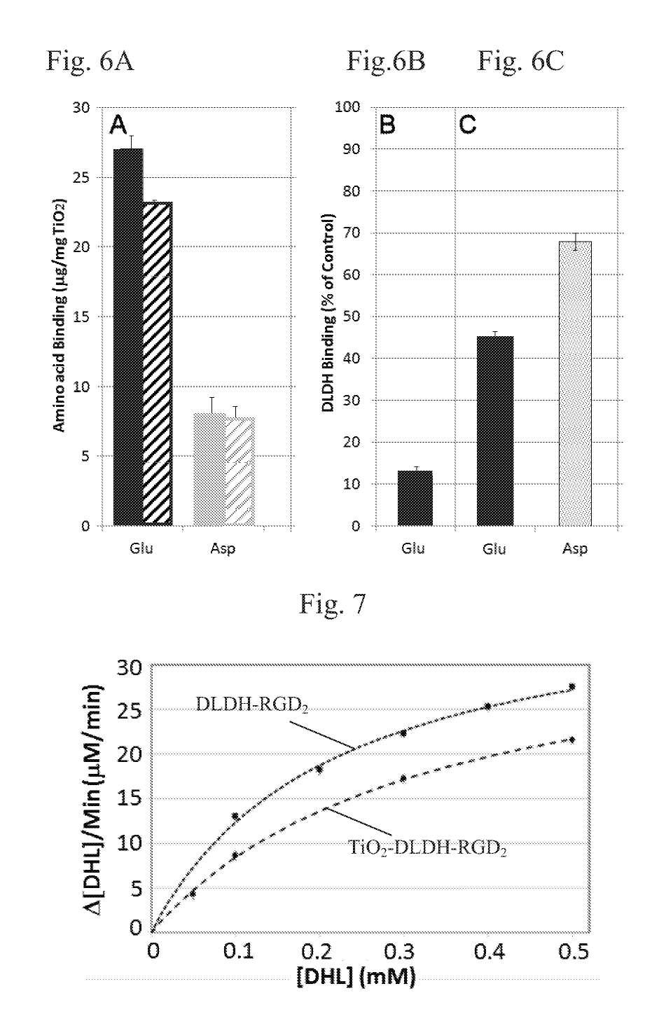

In order to visualize the DLDH-RGD₂ binding to TiO₂ we incubate DLDH-RGD₂ to Rutile plates, and used gold-NP-DLDH-antibody. FIG. 5A shows the gold NP on Rutile plates. A control without DLDH is shown in FIG. 5B.

Inhibitory Effect of Carboxylic Acids on DLDH-RGD₂ Binding to TiO₂

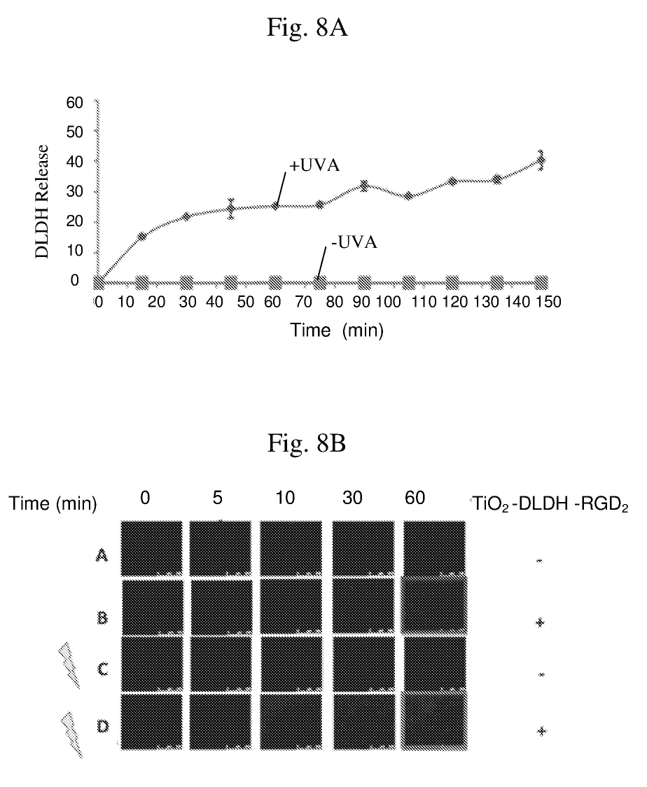

Carboxylic acids, have been shown in the literature to associate with TiO₂ While at low pH their interaction with the oxide is mainly electrostatic, at neutral pH the binding of glutamate is mainly via coordinative bonds while that of aspartate is dramatically reduced. Therefore, we set to study the effect on DLDH-RGD2 binding to TiO₂ of carboxylic amino acids addition to the reaction mixture. As shown in FIG. 6A, glutamate readily binds to TiO₂ (Anatase) at pH 7.5 while aspartate binds much less. This binding was abolished at pH 9.5 (data not shown). The binding capacities obtained for glutamate and aspartate (27.0 and 8.0 mg amino acid per gram of TiO₂, respectively) indicate coverage of 2.23 and 0.66 molecules per square nm for the two amino acids, respectively. Assuming about 10 Ti atoms per nm and binding of one carboxylic acid to two Ti atoms this figure represents close to full coverage of the Ti surface by glutamate and 30% of that for aspartate.

As shown in FIG. 6B, pre-coating of the TiO₂ nanoparticle with DLDH hardly affected glutamate and aspartate binding by the oxide. In contrast, binding of DLDH to glutamate-pre-coated nanoparticles was much reduced which might be expected due to the high coverage of the TiO₂ surface by the amino acid. The ability of glutamate and aspartate to compete with DLDH binding was exemplified next. When each of the amino acids was included in the reaction mixture, concomitantly with DLDH, inhibition of the protein binding to the oxide was observed which was higher for glutamate than for aspartate (FIG. 6C). It is pertinent to note that chemical modification of DLDH by blocking carboxylic groups with carbodiimide resulted in complete abolishment of DLDH binding to TiO₂ (data not shown).

The Enzymatic Activity of DLDH-RGD₂ and TiO₂-DLDH-RGD₂

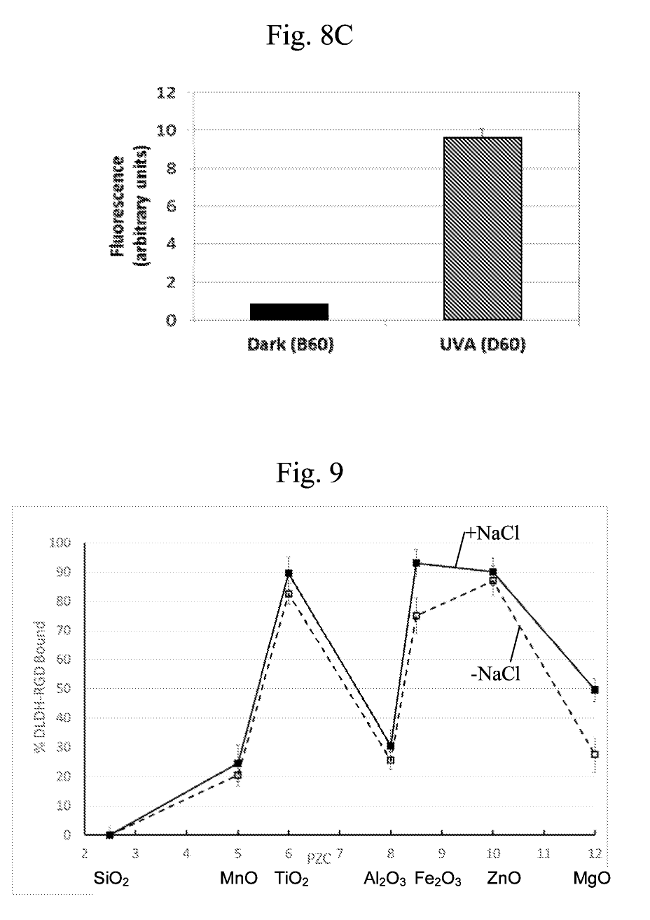

To determine whether DLDH-RGD₂ binding to TiO₂ affects its activity, the enzymatic activities of the TiO₂-adsorbed enzyme with that of the enzyme in solution were compared. Michaelis-Menten curves for dl-dihydrolipoamide (DLH) oxidation by the soluble and the TiO₂-bound DLDH-RGD₂ depicted in FIG. 7 clearly show that the enzyme retains its activity upon binding to TiO₂ with some increase in the apparent Km value (0.215 to 0.327 mM upon TiO₂-binding) and practically no change in kcat (3.55 vs 3.25 sec⁻¹, respectively).

The Effect of UVA Illumination on DLDH-RGD₂ Release from the Complex with TiO₂

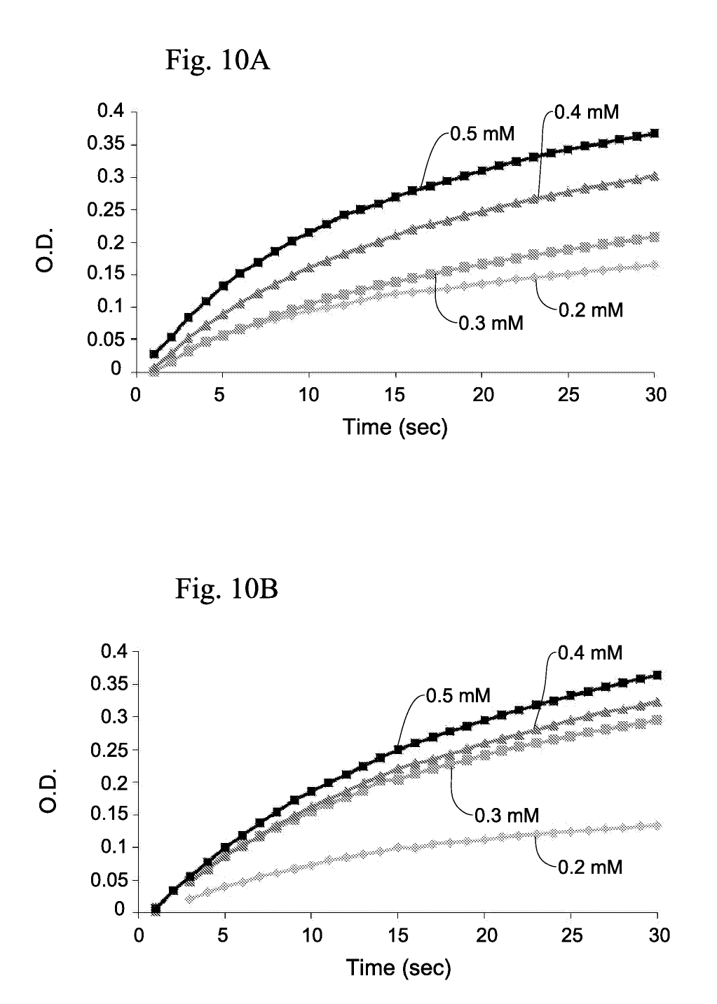

The TiO₂-DLDH-RGD₂ complex is fully stable in vitro in the dark (FIG. 8A). However, when illuminated with UVA at 365 nm it is released, at least in part, from the TiO₂ surface, in a form that retains the enzyme activity and cytotoxicity of the protein. A similar effect was observed when the complex was added to B 16F10 cells. When the cells were kept in the dark the complex remained intact and the protein failed to enter the cell. Upon UVA illumination the protein was released from the complex and penetrated the cells (FIG. 8B). The mean fluorescence intensity in the blue laser channel, representing labeled DLDH was quantified. As shown in FIG. 8C, a marked increase in the insertion of labelled DLDH-RGD₂ into the cells over time under UVA illumination (min) was observed upon UVA illumination, but not in the dark.

Oxide-specificity of DLDH-RGD₂

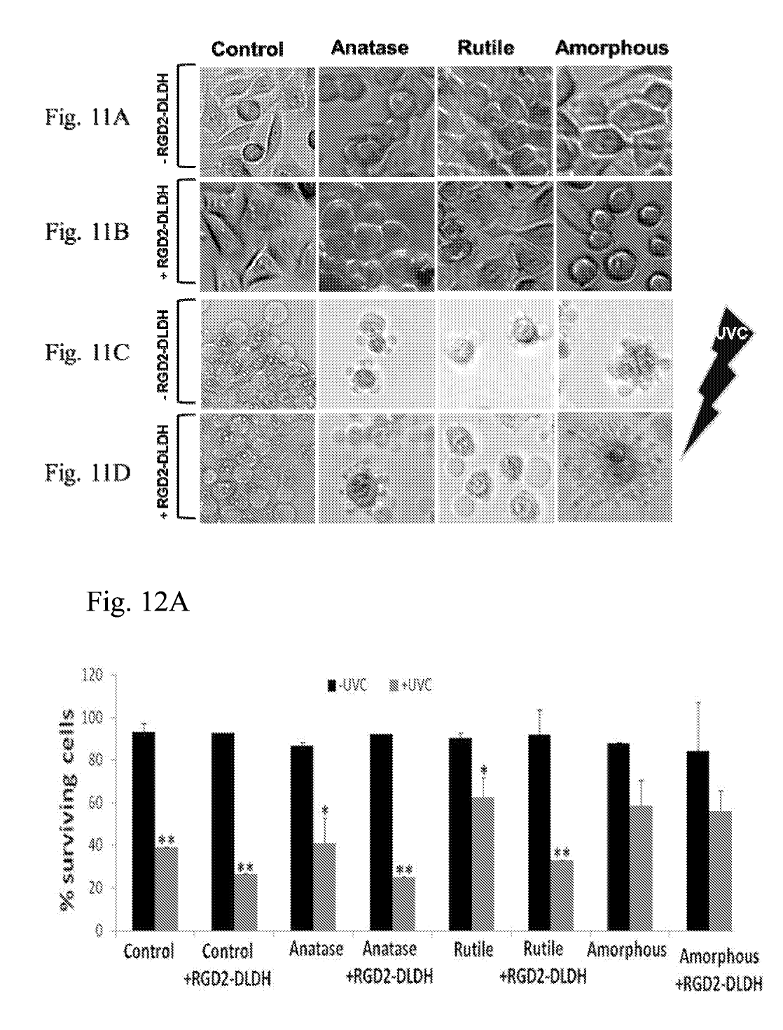

The capability of DLDH-RGD₂ to form complexes with metal oxides other than TiO₂ at pH 8.0 was tested with the acidic SiO₂ and MnO, the amphoteric Al₂O₃ and Fe₂O₃ (magnetite) and the basic ZnO and MgO. Of these, DLDH-RGD₂ was found to also bind magnetite and ZnO. Similarly to TiO₂, the binding was not affected by NaCl presence (FIG. 9).

### 2. Cytotoxic Effect of DLDH-RGD₂ on Cells

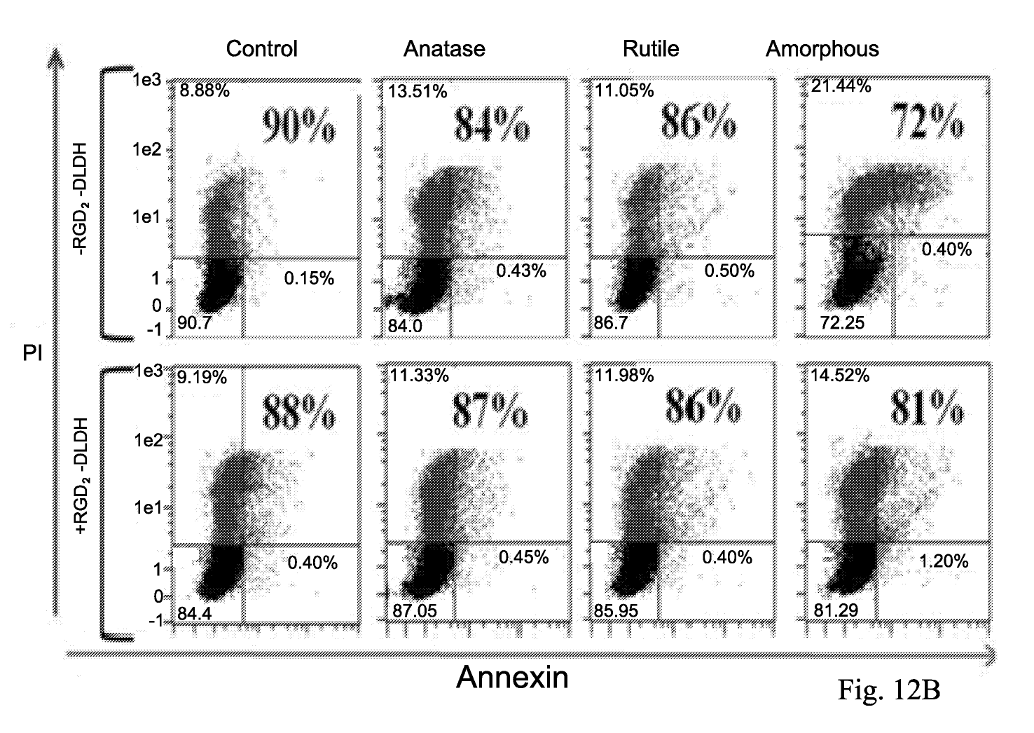

As mentioned in the Experimental section, DLDH-RGD₂ was fluorescently labeled to enable its monitoring in biochemical and confocal experiments. Neither the enzyme activity (FIG. 10) nor the TiO₂ binding capability (data not shown) were affected by the protein labelling.

2.1 The Cytotoxic Effect of TiO₂-DLDH-RGD₂ Under UVA Illumination or In the Dark.
Effect of Different TiO₂ Disks Preparations±DLDH-RDG₂ on the Morphology of HeLa Cells Before/After UVC (254 nm) Illumination

Since in this work we aim at using the combined cytotoxic ROS production effect of TiO₂ and that of DLDH on cancer cells, we next examined the components of the biocomplex (TiO₂, DLDH, RGD) on a selected cancer cell-line (HeLa).

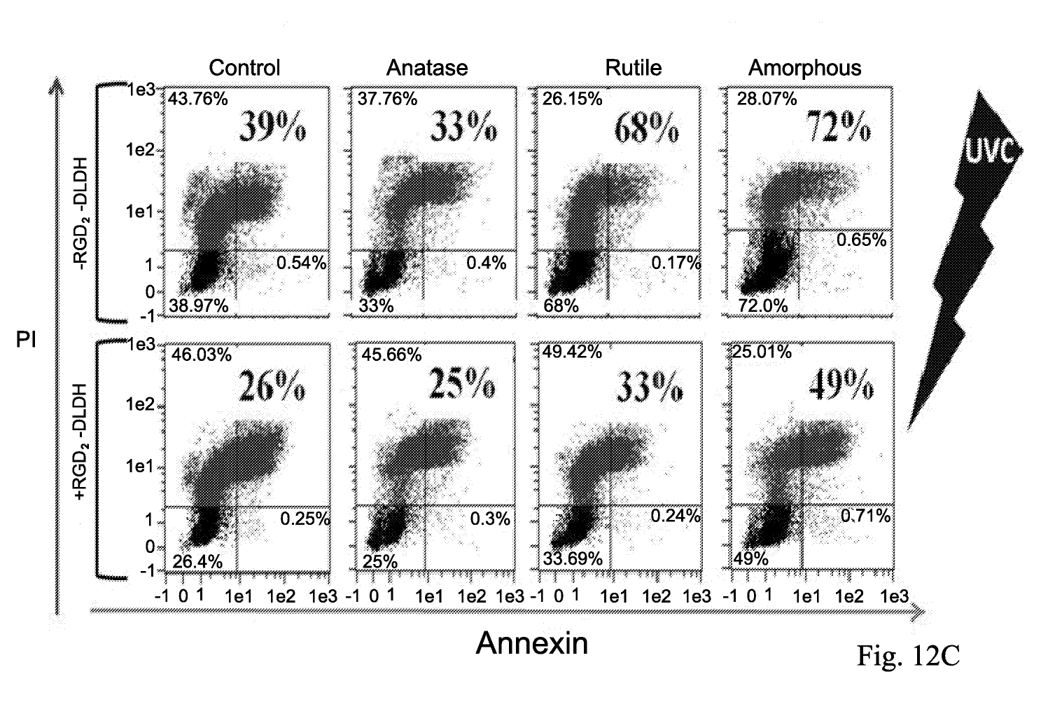

FIGS. 11A-D depicts TiO₂-coated disks (amorphous, rutile and anatase), prepared by thermal treatment of TiO₂ plates (see above). The disks were further coated with DLDH-RGD₂ protein. Uncoated disks served as controls. The disks seeded with HeLa cells (150,000/well) which over-express αvβ₃ integrin. Before illumination, the control cells in the absence (FIG. 11A) or presence (FIG. 11B) of DLDH-RGD₂ protein, maintained classical HeLa morphology and density, the cell phenotype appeared to be more round and condensed. Following UVC illumination, 10 min, blebbing of the cell membrane, as well as a reduction in cell density, a feature of apoptosis, was evident (FIG. 11C). Interestingly, this effect was more significant in the presence of the DLDH-RGD₂ protein (FIG. 11D). After an overnight incubation, nuclear dye (Hoechst) was added and the cells were visualized by fluorescent microscopy.

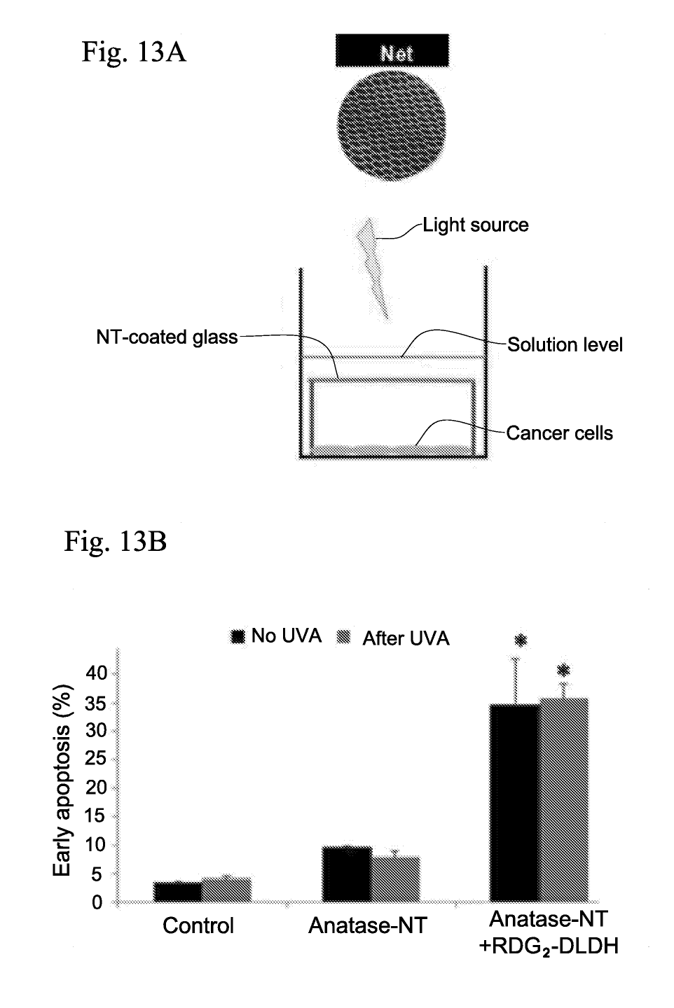

The cells from the same experiment were collected and examined for survival by Annexin-PI (An-/PI-) Fluorescence-activated cell sorting (FACS) analysis. Before Illumination (FIG. 12A, black bars), the cells survival remained high and was similar for the different conditions. Illumination by UVC for 10 minutes (grey bars) induced a differential decrease in survival in comparison to control non-illuminated cells. In the absence of DLDH-RGD₂ protein, two titanium conformations, rutile and amorphous, were least potent (58 and 62% average survival rate, respectively), while control (HeLA) and anatase treated cells exhibited a comparable significant reduction in cell survival (39 and 41%, respectively).

Illumination of DLDH-RGD₂ protein in amorphous preparation had no additional effect on survival. However, the addition of the protein induced a significant effect in rutile disks (39% survival) and more potently in control (26% survival) and cells grown in the presence of anatase disks (25% survival). Representative results before (FIG. 12B) and after UVC illumination (FIG. 12C) are depicted, with the percentage of surviving cells (Annexin-/PI-) in each of the treatments shown in a text box within each graph (*, p<0.05; **, p<0.005). Taken together, these results imply that the titanium binding protein with its integrin-binding-RGD tails autonomously, as well as in combination with anatase TiO₂, sensitizes cell's response to UVC, probably due to its ROS producing ability.

### Effects of Anatase Nanotubes (NT) Glass Matrix and Its Effect±DLDH-RDG₂ Protein on the Survival of HeLa Cells Before/After UVA (365 nm) Illumination

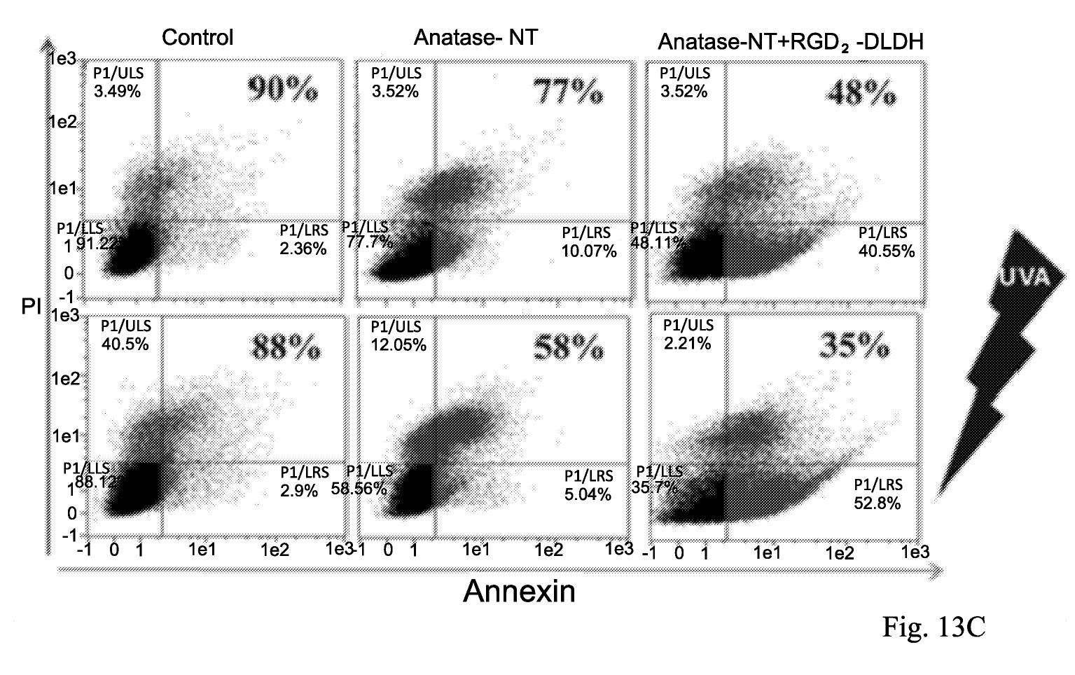

To improve the illumination methods, radiation was switched to UVA light, which is of a longer wavelength (365 nm) in the near UV range. This radiation flux is far below the radiation flux considered safe by International standards and is in addition less mutagenic and more penetrable than UVC. Similar UVA irradiation are currently under use in various skin diseases (Malinowska et al. 2011). The next step was to examine additional matrixes that might be more relevant for our experimental model. Glass cover slips were chosen for further validation. In order to increase surface area and ROS activity of the TiO₂, we have developed 8 nm Ø anatase-NT (see above). The glass plates were prepared ±anatase-NT±DLDH-RGD₂ protein. Uncoated glass cover slips served as controls. HeLa cells (150,000 cells/24 wells) were seeded in the presence of the different preparations (FIG. 13A) and were examined by FACS before and after an hour of UVA illumination for apoptotsis/survival. Before UVA illumination (black bars), no significant apoptosis was observed under the different conditions. However, following an hour of illumination under UVA (grey bars), the titanium produced apoptosis in 18% of cells and a significant synergistic effect was further observed in the presence of the DLDH-RGD₂ protein (44% apoptosis). Representative results (FIG. 13B) before and after UVA are depicted, with the percentage of surviving cells (Annexin-/PI-) in each of the treatments shown in a text box within each graph (*, p<0.05; **, p<0.005). The nets were covered with/without the anatase-NT with/without DLDH-RGD₂ protein in the presence/absence of an hour of UVA illumination (FIG. 13B). No significant apoptosis was observed in control and anatase-NT treated cells in the absence or presence of UVA illumination without DLDH-RGD₂. However, nets covered with anatase-NT and the DLDH-RGD₂ produced a similar induction of apoptosis (35%) without illumination and after UVA illumination. Representative results (FIG. 13C) before and after UVA are depicted, with the percentage of surviving cells (Annexin-/PI-) in each of the treatments shown in a text box within each graph (*, p<0.05).

Exploring the Cytotoxic Effect of TiO₂-DLDH-RGD₂ (NP) in Cancer Cells

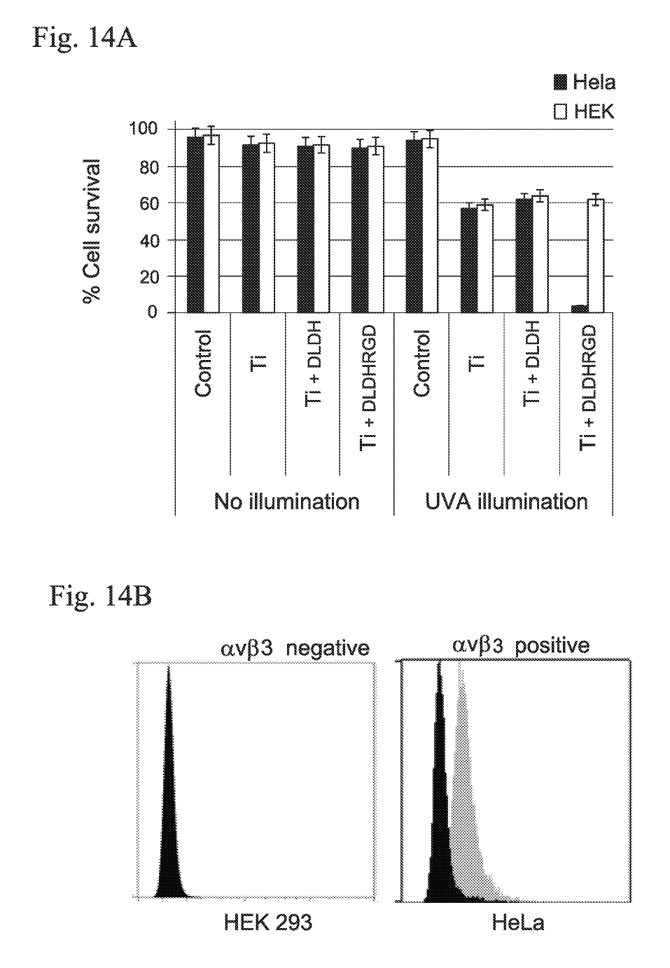

To examine the cytotoxic effect of the nanobiocomplex (TiO₂DLDH-RGD₂) under UVA illumination on cervical cancer cells (Hela) and normal cells (HEK293), the cells incubated (100,000 cells/well) for 1 h under UVA illumination or without, in the presence of TiO₂-DLDH-RGD₂ (NP)-(1 μm). After 48 h incubation (for recovery), cell survival was tested by confocal microscopy and FACS analysis (FIG. 14A). UVA illumination activates the TiO₂ and induces cell death in both cancer (Hela) and normal (HEK293) cells (FIG. 14B). In Hela this effect is enhanced upon TiO₂-DLDH-RGD₂ addition (in B16F10 only). No such effect was observed without UVA illumination.

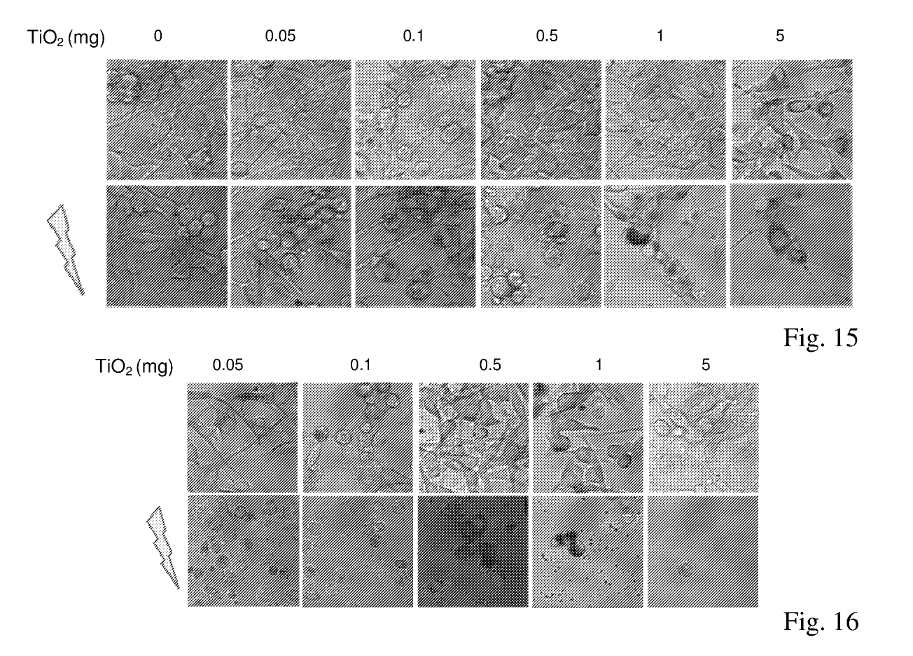

FIG. 15 shows the cytotoxic effect is observed after 48 h incubation in the presence of increasing concentrations of A-NP (0.05-5 mg/ml) following UVA illumination (1 h). The cytotoxic effect is evident by a reduction in cell density—mouse melanoma cell line (B16F10).

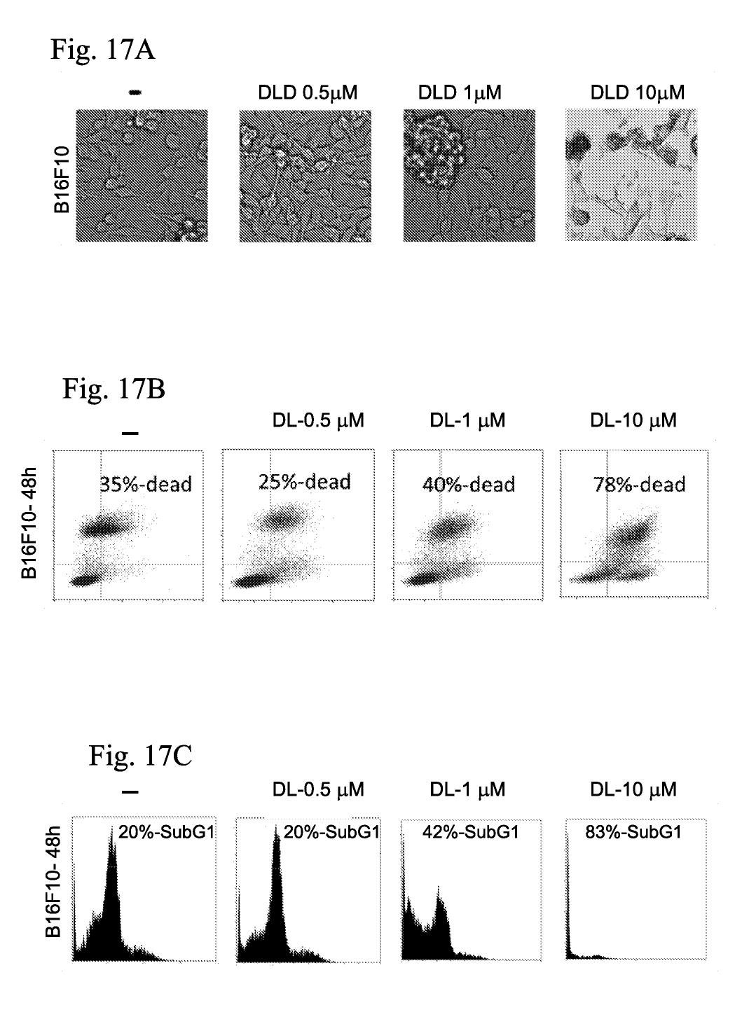

This experiment was repeated with the addition of 0.5 mg of fluorescamine-labeled DLDH-RGD₂ and different amounts of A-NP (0.05-5 mg/ml). The cell nuclei, stained by Draq5 are indicated by a red color, whereas the fluorescamine-labelled protein is in blue. The cells were incubated for 1 h either in the dark or under UVA illumination. While the cells remained intact in the dark a substantial amount of nuclear shredding, indicative of apoptosis, was shown with increasing TiO₂ concentrations (FIG. 16). These results indicate the requirement of UVA illumination for the nanobiocomplex activation.

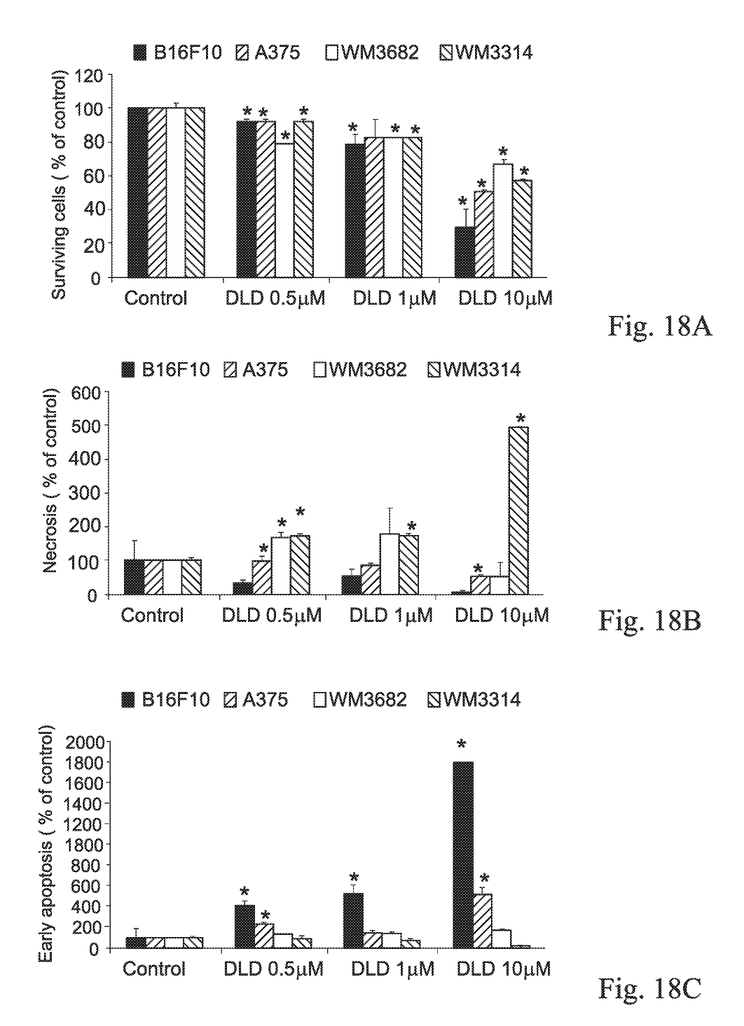

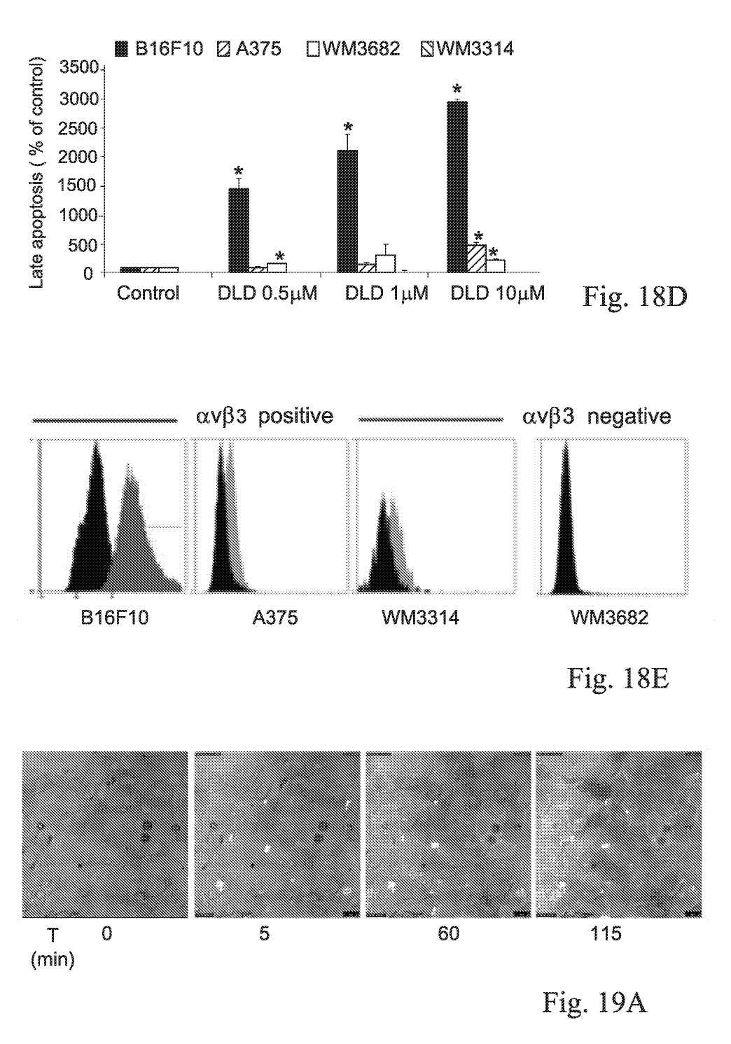

As shown in FIG. 17A, incubation for 48 h at 37° C. of DLDH-RGD₂ at increasing amounts concentration (0.5-10 μM) with 100,000 mouse melanoma cell line (B16F10) leads to incorporation of the protein into the cells and initiation of an apoptotic effect. FACS analysis (FIG. 17B) showed increased early and late apoptosis which occur with increasing DLDH concentration. The percentage of cells in SubG1 (FIG. 17C) increases from 20% to 83%. Incubation of the cells with DLDH without RGD tails resulted in a similar but slower effect. In contrast, ovalbumin, served as a control, failed to enter the cells and cause cell death. FACS analysis was repeated with four different human melanoma cell lines (FIG. 18A and B).

2.2 The Cytotoxic Effect of DLDH-RGD₂ (in the Absence of TiO₂) in B16F10 Melanoma cells, Under UVA Illumination or in the Dark.

It was assumed that this phenomenon was due to an excess of unbound protein (DLDH-RDG₂).

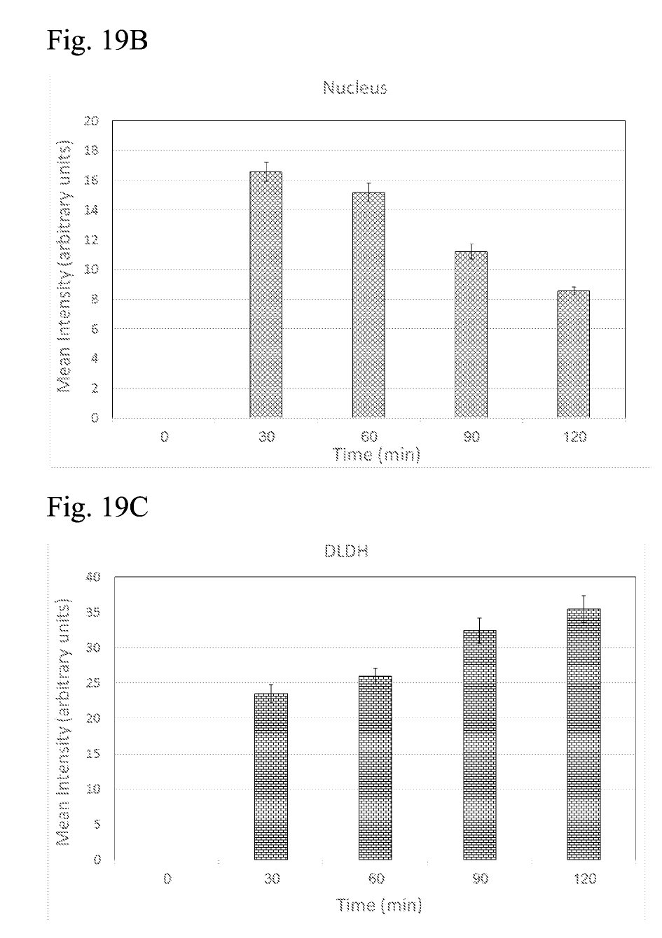

In FIG. 19A, confocal images (time-laps video) of an in-situ apoptosis assay in B 16F10 melanoma cells (100,000 cells/well) that were incubated with 5 μm DLDH-RGD₂ during time (min), without illumination, are shown. The assay follows in multiplex the cell nuclei, which is stained by Draq5 (far red), Fluorescamine-labeled DLDH-RGD₂ (blue), Psiva (green), indicative for early apoptosis and PI (pink) is indicative for late apoptosis. It is noticeable that DLDH-RGD₂ enters the cytoplasm within 5 min leading quickly to the destruction of the cell nucleus.

The mean fluorescence intensity of the each laser channel was measured during time. FIG. 19B shows the disappearance of the nuclei that points to cell death, FIG. 19D shows the insertion of fluorescamine-label DLDH-RGD₂ into the cells, in FIG. 19C the increase of the PI dye that detects late apoptotic death is shown and in FIG. 19E the increase of the Psiva dye that detects early apoptotic death is shown.

By comparing the cytotoxic effect on mouse melanoma cell line (B16F10) of TiO₂-DLDH-RDG₂ with that of DLDH-RGD₂ it is obvious that the complex requires UVA illumination to be effective, while the protein alone acts on the cells also in the dark. Considering the data in FIG. 3 showing that ROS production by DLDH-RGD₂ is independent of UVA illumination, led us to hypothesize that UVA activation is needed to release the protein from the complex in situ. To examine this possibility we subjected the release of DLDH-RGD₂ from the complex TiO2-DLDH-RDG₂ upon UVA illumination in cell free conditions (FIG. 8).

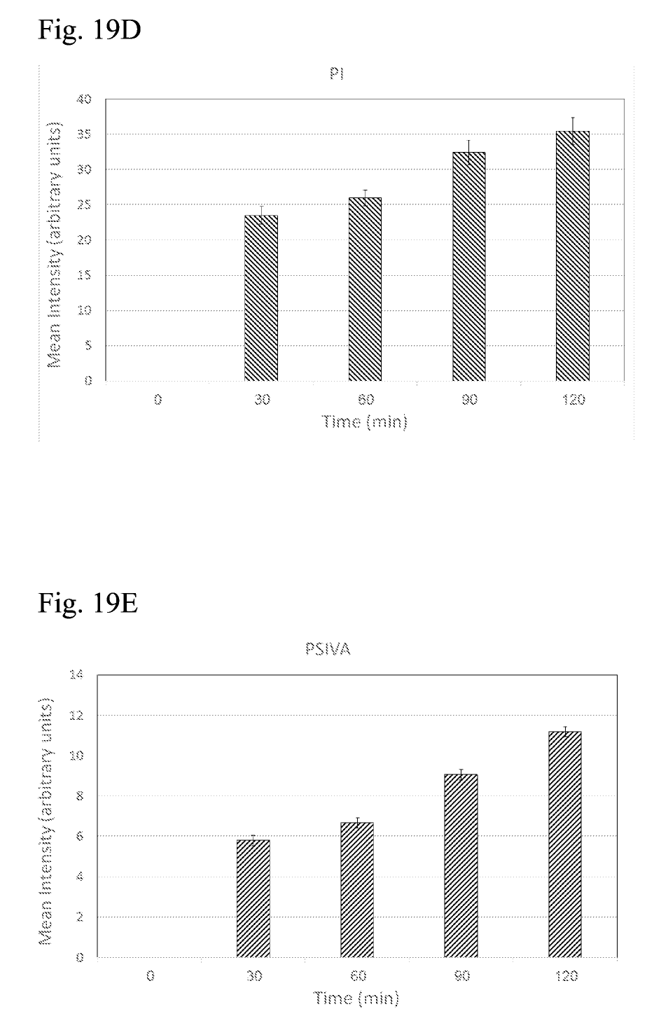

The cytotoxic effect of DLDH-RGD₂ on melanoma cells compared to normal cells was investigated next. FIG. 20A depicts the cytotoxic effect of Fluorescamine-labelled DLDH-RGD₂ (5 μM) upon incubation with HEK293 (normal kidney cells) and B16F10 cells (100,000 cells/well), for 6-48 hrs. The cell nuclei were stained by Draq5 (red). As shown in the Figure, no cytotoxic effect was observed with the HEK293 cells for at least 24 h, apparently due to its low integrin expression. In contrast, B16F10 cells which highly express the integrin, as expected, were susceptible to DLDH-RGD₂ showing high cytotoxicity within 6 h of application. FIG. 20B shows cell membrane αvβ3 integrin expirations by FACS analysis.

### 2.3 The Effect of the RGD Tails on the Cytotoxicity of DLDH

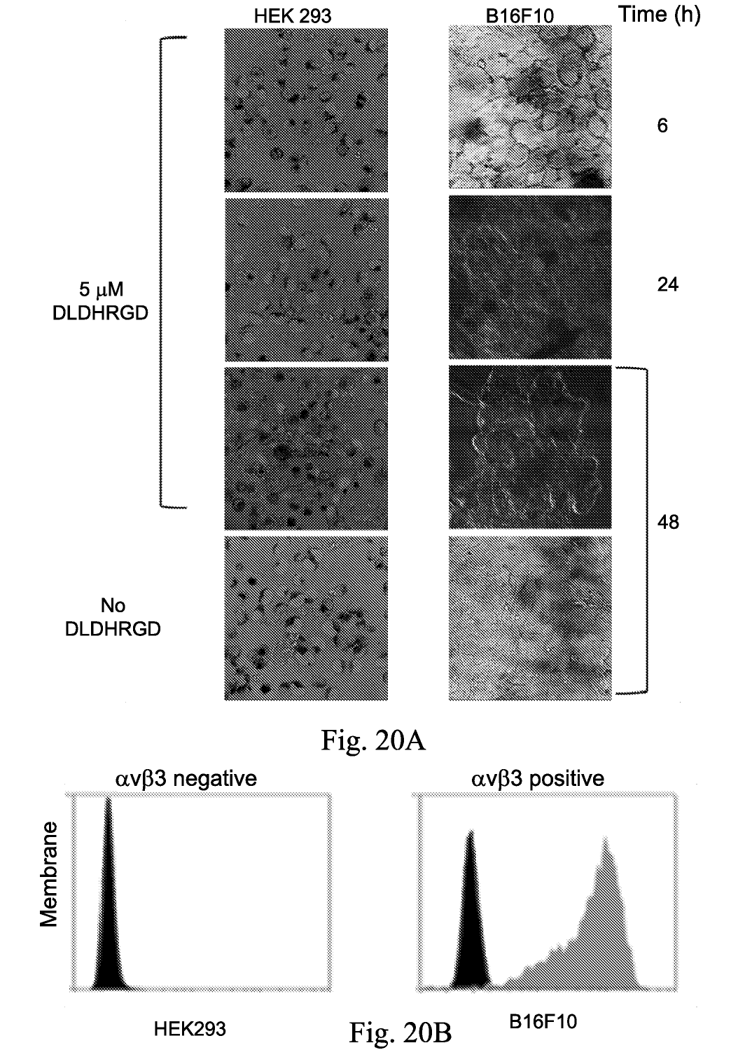

Next, the inventors compared the contribution of the RGD tails to the DLDH protein cell penetration. B16F10 (100,000 cells/well) were seeded overnight. Fluorescamine-labelled DLDH (FIG. 21A) or DLDH-RGD₂ (FIG. 21B) were added to each well and the cytotoxicity was visualized under Leica SP8. The solvent served as negative control (FIG. 21C). The cells nucleauses were stained by Draq5 (Far red), and protein stained by Fluorescamine (blue). Results indicated that protein penetration to the cells was enhanced in the presence of the RGD tails (FIG. 21B), leading to nuclear destruction and cell death.

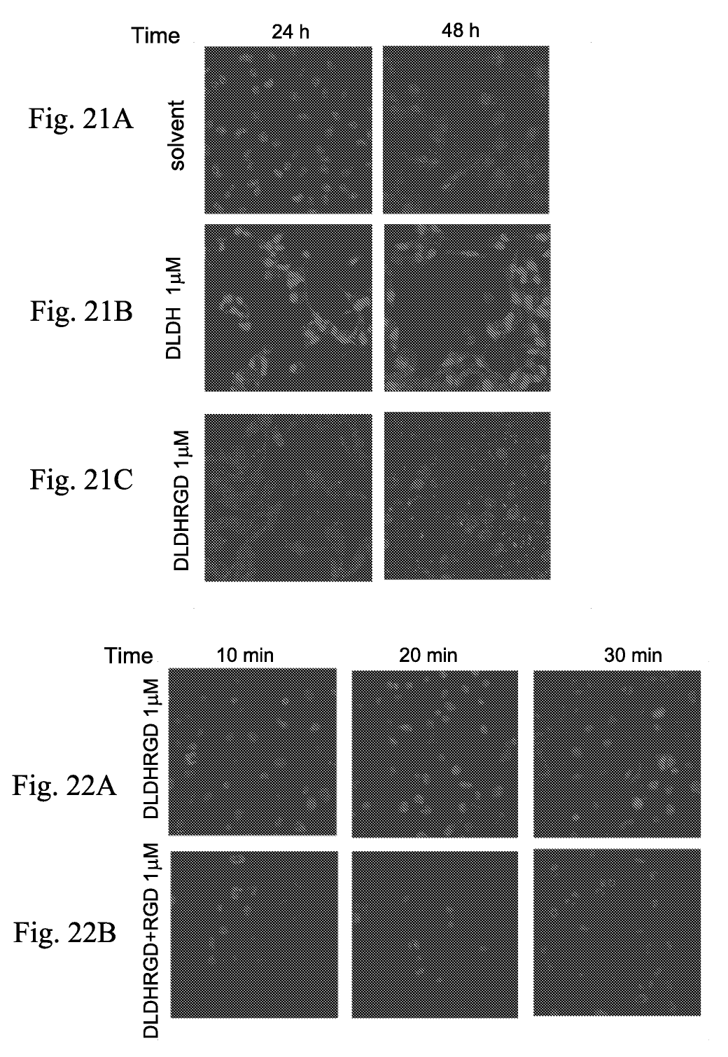

Next, it was examined whether RGD tri-peptides inhibit DLDH penetration to the cells. HEK 293 cells (normal kidney cells) were transfected to express the integrin αvβ₃ (HEK293B3). 100,000 cells/well were seeded overnight. Fluorescamine-labelled DLDH±RGD (1 μM) was add to each well in presence (1 mM) or absence of RGD (as a competitive inhibitor) and the penetration rate as well as the cytotoxicity were determined by confocal Microscopy (Leica SP8). The cells nuclei was stained by Draq5 (red). Blue marks the Fluorescamine labelled DLDH-RGD₂. As shown in FIG. 22, while in the absence of RGD addition, DLDH quickly incorporated into the cells, inducing cell death as previously shown (FIG. 22A), while co-incubation of the protein in the presence of RGD (1 mM), which act as αvβ₃ antagonist, potently prevented DLDH-RGD₂ from entering the cells (FIG. 22B). These results further indicate the integrin mediated nature of DLDH-RGD₂ take in by the cancer cells.

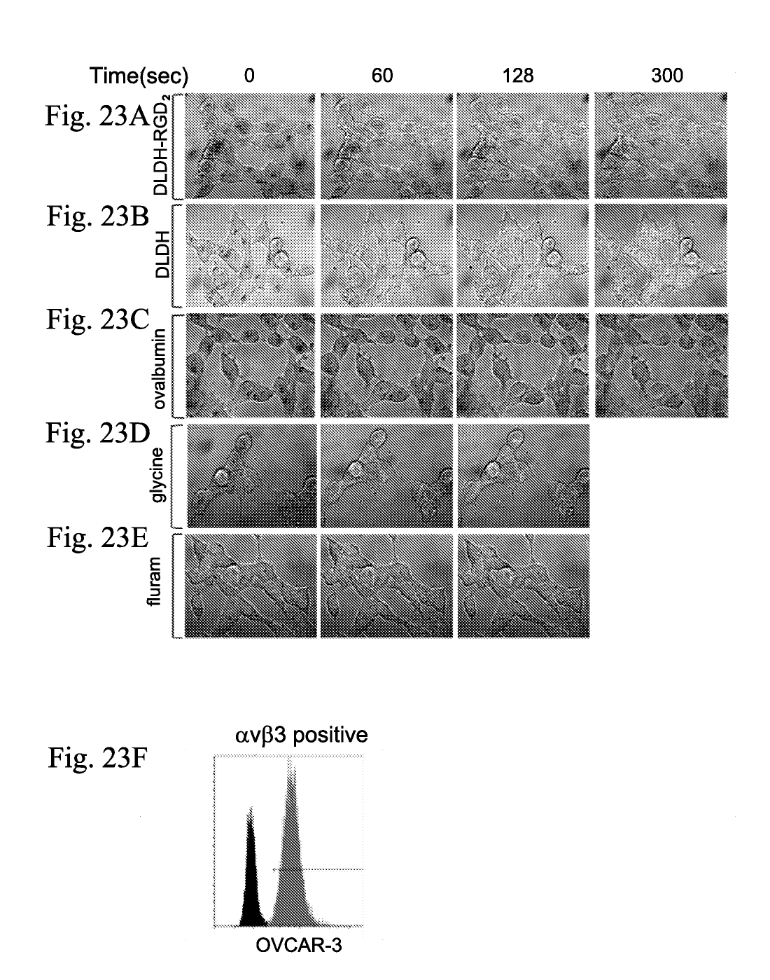

In FIG. 23 the intake of fluorescamine(Fluram)-labelled DLDH-RGD₂, DLDH (devoid of RGD tails) and Ovalbumin and glycine, as a negative controls, are shown. The nuclei of the selected cancer cell-line (OVCAR3, ovarian) were stained bleu using DraQ5. Results show that DLDH-RGD₂ is the first to enter the cells (37 sec, probably by endocytosis) and reach out-side of the nucleus (60 sec), leading to apoptotic cell blebbing (83 sec) and collapse (128sec), which not occur at the controls. The experiment was videoed during 370 sec at LSM 510 META (X63).

DLDH-RGD₂ was shown to incorporate the cancer cells significantly faster than DLDH. However, the time interval from cell entry till cell death (23 sec to apoptosis and additional 45 sec the cell collapse) was comparable. The controls, ovalbumin and glycine entered the cells but did not initiate apoptosis process (Table 3).

TABLE 3
comparison between the complex
components during incubation time (sec).

| Function | DLDH-RGD₂ | DLDH | OV |
|---|---|---|---|
| Incorporation | 37 | 68 | 83.5 |
| into the cell | | | |
| Apoptosis | 83 | 136.5 | X |
| Cell destruction | 128 | 182 | X |

2.4 DLDH as a DNA degrading enzyme

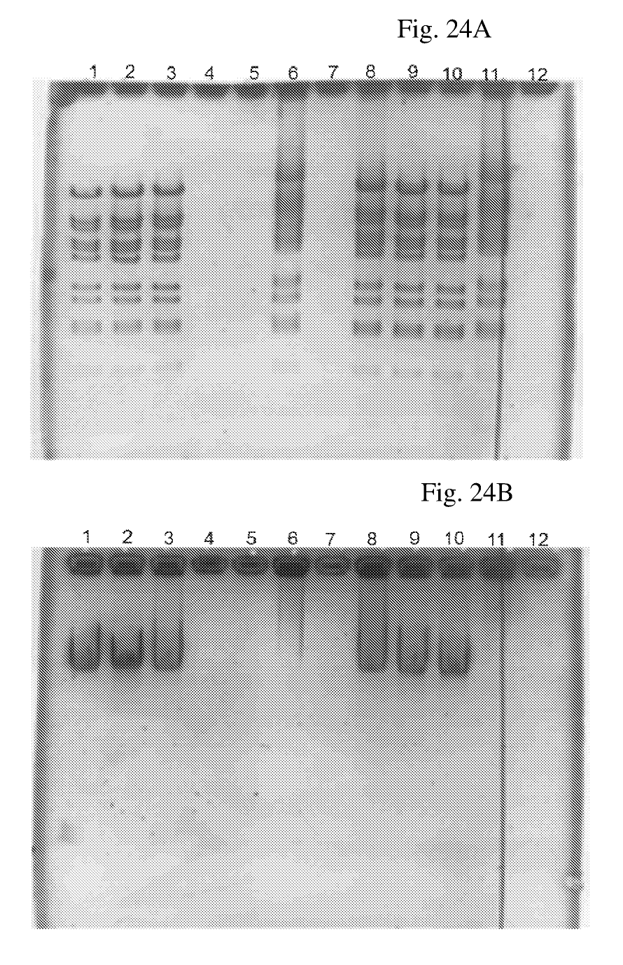

The nucleus destruction by DLDH led us to examine the possibility that the enzyme possesses an activity of DNase. Such an activity is shown in FIG. 24. When DLDH (1 or 10 μg) was incubated with X phage (0.15 μg), degraded (FIG. 24A) or intact (FIG. 24B). Degradation of the phage by DLDH was observed only in the presence of its substrates and was more pronounced at the higher enzyme concentration (lanes 6 and 11).

1.-25. (canceled)

26. A pharmaceutical composition comprising dihydrolipoamide dehydrogenase (DLDH), or a DLDH-based material, for use in medicine, wherein the DLDH-based material is selected from the group consisting of
a. DLDH associated with at least one peptide,
b. DLDH complex with at least one metal or metal oxide, and
c. DLDH associated with at least one peptide as a complex with at least oen metal or metal oxide.

27. (canceled)

28. The pharmaceutical composition according to claim 26, for the treatment of a proliferative disease or disorder.

29. A peptide-modified material comprising DLDH and at least one integrin binding domain.

30. The peptide-modified material according to claim 29, wherein the integrin binding domain is arginine-glycine-aspartic acid (RGD).

31. The peptide-modified material according to claim 29, being associated with at least one metal or metal oxide.

32. The peptide-modified material according to claim 30, wherein the at least one RGD is associated with DLDH through at least one of the N terminus of the DLDH and the C terminus of the DLDH.

33. The peptide-modified material according to claim 30, being in the form of DLDH-RGD₂.

34. A pharmaceutical composition comprising a peptide-modified material according to claim 29.

35. A method for treatment or prevention of a disease or disorder, the method comprising administering to a subject in need thereof an effective amount of DLDH or a DLDH-based material, wherein the DLDH-based material is selected from
a. DLDH-associated with at least one peptide,
b. DLDH complex with at least one metal or metal oxide, and
c. DLDH associated with at least one peptide as a complex with at least one metal or metal oxide.

36. A photodynamic therapeutic method comprising administering to a subject in need thereof an effective amount of a photo-reactive DLDH-based material selected from DLDH-RGD, DLDH-TiO₂ and DLDH-RGD₂-TiO₂, and irradiating said subject or a region of the subject's body in order to render active said photo-reactive material.

37. A composition comprising a peptide comprising at least one CHED motif and a pharmaceutically acceptable carrier.

38. The composition according to claim 37, wherein the CHED motif is associated with a metal or a metal oxide.

39. The method of claim 35, wherein the DLDA-based material comprises DLDH complex with TiO₂.

40. The method of claim 35, wherein the DLDA-based material comprises DLDH associated with RGD₂ and complexed with TiO₂.
